(19) United States
(12) Patent Application Publication
(10) Pub. No.: US 2025/0288607 A1
(43) Pub. Date: Sep. 18, 2025

(54) METHODS FOR TREATING SEXUAL DYSFUNCTION ASSOCIATED WITH POLYCYSTIC OVARY SYNDROME (PCOS) OR WITH HYPOACTIVE SEXUAL DESIRE DISORDER (HSDD)

(71) Applicants: INSTITUT NATIONAL DE LA SANTÉ ET DE LA RECHERCHE MÉDICALE, Paris (FR); CENTRE HOSPITALIER UNIVERSITAIRE DE LILLE, Lille (FR); UNIVERSITÉ DE LILLE, Lille (FR)

(72) Inventors: Paolo GIACOBINI, Lille Cedex (FR); Vincent PREVOT, Lille Cedex (FR); Konstantina CHACHLAKI, Lille Cedex (FR); Mauro Sergio Batista DA SILVA, Boston, MA (US)

(21) Appl. No.: 18/862,779

(22) PCT Filed: May 12, 2023
(86) PCT No.: PCT/EP2023/062763

§ 371 Date: Nov. 4, 2024

(30) Foreign Application Priority Data
May 13, 2022 (EP) 22305707.6

(51) Int. Cl. A61K 33/00; A61K 31/198; A61K 45/06; A61P 15/00

(52) U.S. Cl. CPC A61K 33/00; A61K 31/198; A61K 45/06; A61P 15/00

(57) ABSTRACT

In the present invention, inventors demonstrate that prenatal excess of anti-Müllerian hormone triggers PCOS-like impairment in female sexual behavior in mice. Sexual dysfunction in PCOS-like mice is associated with decreased expression of neuronal nitric oxide synthase (nNOS) neurons in different hypothalamic regions known to be involved in female sexual behavior: rostral periventricular area of the third ventricle (RP3V), ventromedial nucleus of the hypothalamus (VMH), and arcuate nucleus (ARN) during estrus. Chemogenetic inhibition of nNOS neuronal activity in the ventromedial nucleus of the hypothalamus of control adult females recapitulates PCOS-like sexual dysfunction. Of clinical relevance, administration of nitric oxide (NO) donor rescues normal sexual behavior in PCOS-like mice. Accordingly, the present invention relates to invention relates to Nitric Oxyde (NO) agent for use in the prevention or the treatment of sexual dysfunction associated with Polycystic Ovary Syndrome (PCOS) or with Hypoactive Sexual Desire Disorder (HSDD) in a subject in need thereof.

### FIELD OF THE INVENTION

The present invention relates to a method of preventing or treating sexual dysfunction associated with Polycystic Ovary Syndrome (PCOS) in a subject in need thereof.

### BACKGROUND OF THE INVENTION

Polycystic ovary syndrome (PCOS) is a highly prevalent disease affecting 5-18% of women of reproductive age worldwide (1, 2). PCOS is diagnosed upon the presence of at least two out of the three prime features: high circulating levels of androgens (hyperandrogenism), menstrual irregularities (oligo-anovulation), and polycystic-like ovarian morphology (2, 3).

Beyond its implications leading to female infertility, the disease is associated with several metabolic disruptions, cardiovascular diseases, and psychosocial disorders (4). Among these neurological implications, it has become clear that about 30% or more of PCOS patients suffer from sexual dysfunctions, with clinical studies reporting a high risk of low sexual arousal, desire and satisfaction, and impaired lubrication and orgasm (5-9). These symptoms allude to disturbances in brain circuits controlling sexual function in the context of PCOS.

Neural circuits driving female sexual behaviours are conserved among vertebrate species operating under the influence of sex steroid hormone modulation, which is paramount for partner interaction, receptivity, and sexual performance (10, 11). Indeed, gonadal sex hormones are implicated in shaping circuit architecture in the hypothalamus during development and activating these same neonatally programmed circuits over reproductive adult life in many species (12-16). The hypothalamus integrates sensorial stimuli and autonomic arousal from endogenous sex drive cues (e.g.: estrous phase, energy status, hormone milieu, genital stimulation) to convey this information to other brain areas and peripheral nerves (10, 17, 18). The ventromedial nucleus of the hypothalamus (VMH) is considered to be the hub of specialized neurons with intrinsic properties driving different components of sexual behavior (19-22). The VMH harbors neurons expressing the neuronal nitric oxide synthase (nNOS), the enzyme responsible for the production of nitric oxide (NO), a key gaseous neurotransmitter that stimulates female sexual behavior (23, 24) and communicates with other circuits within the social brain (25, 26). Despite current advances unraveling novel pathways in the female sexual brain with specific behavioral responses, there is a clear lack of knowledge on how disturbances in these circuits may participate in sexual dysfunctions affecting one-third of women with PCOS.

Growing evidence indicates that androgen excess in utero induces a developmental reprogramming of the female fetal brain toward the manifestation of PCOS traits later in life (27-30). Some studies suggested that the clinical signs of hyperandrogenism have detrimental sexual effects (5) suggesting a negative correlation between androgen levels and sexual function in PCOS. In recent years, it has been proposed that prenatal AMH excess may trigger gestational hyperandrogenism via the inhibition of placental aromatase (30, 31), and that women with PCOS display higher circulating levels of androgens and AMH during pregnancy as compared to healthy women (30, 32, 33). Prenatal AMH treated (PAMH) mice reliably recapitulate all the mouse equivalents of the PCOS Rotterdam criteria (30, 34) and are thus a preclinical model to mimic the human PCOS condition. PAMH female mice also display pronounced neuroendocrine dysfunction leading to exacerbated luteinizing hormone (LH) secretion (30) as in PCOS women (35, 36), denoting the presence of prenatally-reprogramed defects within the gonadotropin-releasing hormone (GnRH) neuronal network. Thus, prenatally AMH excess-mediated derangements in the female brain might be key to understanding the pathophysiology of PCOS.

Here, inventors interrogated whether prenatal AMH excess could underpin defects in sex circuits promoting sexual dysfunction in PCOS-like female mice. They uncovered a profound decrease of nNOS and progesterone receptor (PR) expression in the VMH associated with significant impairment of sexual receptivity.

Nevertheless, normal sexual function in PAMH female mice is restored to control levels upon peripheral injection of NO donor. Moreover, the inventors found that PCOS-like females present a lower percentage of cFOS-expressing cells in several hypothalamic and limbic structures involved in the control of sexual behavior as compared with control groups. NO donor-treatment restored normal neuronal activation in those regions, indicating that NO-mediated amelioration of sexual dysfunction in PAMH female mice may result from the recovery of normal neuronal activation in hypothalamic and limbic structures in the female brain involved in sexual behavior.

Performing a series of acute functional manipulations in freely moving females, inventors show that chemogenetic silencing of nNOS^{VMH} neurons in control females recapitulates PCOS-like sexual dysfunctions. Taken together, they unveil a brain pathway potentially underpinning the etiology of low sexual drive in PCOS, while pointing to prospective therapeutic approaches to rescue normal sexual function in these women and potentially in other forms of low sexual desire, such as hypoactive sexual desire disorder (HSDD), the most severe form of low sexual drive (see Nappi R E, et al. Int J Womens Health. 2010 Aug. 9; 2:167-75. and Lindau S T, et al., N Engl J Med. 2007; 357 (8): 762-774).

### SUMMARY OF THE INVENTION

An object of the invention relates to Nitric Oxyde (NO) agent for use in the prevention or the treatment of sexual dysfunction associated with Polycystic Ovary Syndrome (PCOS) in a subject in need thereof.

An additional aspect of the invention relates to a Nitric Oxyde (NO) agent for use in the prevention or the treatment of sexual dysfunction associated with hypoactive sexual desire disorder (HSDD) in a patient in need thereof.

### DETAILED DESCRIPTION OF THE INVENTION

In the present invention, inventors demonstrate that prenatal excess of anti-Müllerian hormone triggers PCOS-like impairment in female sexual behavior in mice. Sexual dysfunction in PCOS-like mice is associated with decreased expression of neuronal nitric oxide synthase (nNOS) neurons in in different hypothalamic regions known to be involved in female sexual behavior: rostral periventricular area of the third ventricle (RP3V), ventromedial nucleus of the hypothalamus (VMH), and arcuate nucleus (ARN) during estrus.

Chemogenetic inhibition of nNOS neuronal activity in the ventromedial nucleus of the hypothalamus of control adult females recapitulates PCOS-like sexual dysfunction. Of clinical relevance, administration of nitric oxide (NO) donor rescues normal sexual behavior in PCOS-like mice.

### Method of Treatment

According an object of the invention relates to a Nitric Oxyde (NO) agent for use in the prevention or the treatment of sexual dysfunction associated with Polycystic Ovary Syndrome (PCOS) in a subject in need thereof.

According to the invention, the term “subject” denotes a mammal, such as a rodent, a feline, a canine, or a primate. In some embodiments, the subject is a human. In some embodiments, the subject is a woman. Particularly, the subject denotes a human with a Polycystic Ovary Syndrome (PCOS). As used herein, the term “subject” encompasses the term “patient”.

As used herein, the term “PCOS” or “Polycystic Ovary Syndrome (PCOS)” is a highly prevalent disease affecting 5-18% of women of reproductive age worldwide (1, 2). PCOS is diagnosed upon the presence of at least two out of the three prime features: high circulating levels of androgens (hyperandrogenism), menstrual irregularities (oligo-anovulation), and polycystic-like ovarian morphology (2, 3). Beyond its implications leading to female infertility, the disease is associated with several metabolic derangements, cardiovascular diseases, and psychosocial disorders (4). Among these neurological implications, it has become clear that about 30% or more of PCOS patients suffer from sexual dysfunctions, with clinical studies reporting a high risk of low sexual motivation and desire, lack of libido and satisfaction, and impaired lubrication and orgasm (5-9). These symptoms allude to disturbances in brain circuits controlling sexual function in the context of PCOS.

PCOS has a strong heritable component (Crisosto et al., 2007; Gorsic et al., 2019; Gorsic et al., 2017), as witnessed by the fact that ˜60-70% of daughters born to women with PCOS will eventually manifest the disease (Crisosto et al., 2019; Risal et al., 2019). In line with that, a recent study showed that daughters of mothers with PCOS have a fivefold-increased risk of being diagnosed with PCOS later in life (Risal et al., 2019). It has been suggested that environmental factors, such as excessive androgen (Abbott et al., 2002; Franks and Berga, 2012; Padmanabhan and Veiga-Lopez, 2013; Risal et al., 2019; Walters et al., 2018b), or elevated levels of anti-Müllerian hormone (AMH) exposure (Tata et al., 2018), may be in part responsible for the development of PCOS.

In particular embodiments, the subject of the present invention suffers from PCOS and/or have been previously diagnosed (or one its parents) with PCOS.

In the context of the present invention, the objective is to treat and prevent PCOS's neurological symptoms especially sexual dysfunctions in PCOS patient, such as low sexual arousal, desire, lubrication, orgasm, and satisfaction. These symptoms refer to disturbances in brain circuits controlling sexual function in the context of PCOS especially and as demonstrated by the inventors, decreased expression of progesterone-sensitive neuronal nitric oxide synthase (nNOS) neurons in the hypothalamus.

In particular embodiments, sexual dysfunctions associated with PCOS is selected from the list consisting of low sexual arousal, desire, lubrication, orgasm, and satisfaction.

The term “Nitric oxide” also called “nitrogen monoxide” or “NO” has its general meaning in the art refers to a chemical compound formed by an oxygen atom and a nitrogen atom. It is a gas under normal conditions of pressure and temperature. In mammals including humans, nitric oxide is a signaling molecule involved in several physiological and pathological processes (Hou, Y C; et al (1999). Current Pharmaceutical Design. 5 (6): 417-41). Nitric oxide is a powerful vasodilator with a half-life of a few seconds in the blood. Standard pharmaceuticals such as nitroglycerine and amyl nitrite are precursors to nitric oxide. Low levels of nitric oxide production are typically due to ischemic damage in the liver. It is an important neurotransmitter in mammals and the only known gaseous neurotransmitter. It is released by endothelial cells, macrophages, liver cells, neurons and many other cells during cardiovascular and tumor pathologies. In the body, nitric oxide is naturally synthesized from L-arginine and oxygen by several enzymes called NO synthases (NOS).

Regarding the use of nitric oxide as a medication, in the European Union (see “Inomax EPAR”. European Medicines Agency (EMA). Retrieved 29 May 2020) nitric oxide in conjunction with ventilatory support and other appropriate active substances, is indicated:
for the treatment of newborn infants ≥34 weeks gestation with hypoxic respiratory failure associated with clinical or echocardiographic evidence of pulmonary hypertension, in order to improve oxygenation and to reduce the need for extracorporeal membrane oxygenation;
as part of the treatment of peri- and post-operative pulmonary hypertension in adults and newborn infants, infants and toddlers, children and adolescents, ages 0-17 years in conjunction to heart surgery, in order to selectively decrease pulmonary arterial pressure and improve right ventricular function and oxygenation.

In the United States it is indicated to improve oxygenation and reduce the need for extracorporeal membrane oxygenation in term and near-term (>34 weeks gestation) neonates with hypoxic respiratory failure associated with clinical or echocardiographic evidence of pulmonary hypertension in conjunction with ventilatory support and other appropriate agents. Nitric oxide was approved for medical use in the United States in December 1999 and for medical use in the European Union in 2001.

In a particular embodiment and in the context of the present therapeutic method of the invention, the nitric oxide (NO) agent is
Inhaled nitric oxide (iNO); or
nitric oxide donor (NO donor).

Such iNO may include gases comprising nitric oxide, such as nitric oxide in a diluent or carrier gas such as nitrogen or helium. The NO-containing gas may be provided by any known method, such as from a gas cylinder or chemically generating the NO at or near the place of administration. The NO-containing gas may be at a higher concentration in the cylinder or other gas source and be diluted to a delivery concentration prior to use. The drug may be provided by a drug delivery device.

The term “NO donor” or “Exogenous NO” (NO-delivery drugs) in the context of the present invention means any compound (biological or chemical) capable of increasing the production of the nitric oxide.

Exogenous NO sources constitute a powerful way to supplement NO when the body cannot generate enough for normal biological functions (Hou, Y. C et al (1999). Curr. Pharm. Des. 5 (6): 417-471). Certain endogenous compounds can act as NO-donors or elicit NO-like reactions in vivo. Nitroglycerin and amyl nitrite serve as vasodilators because they are converted to nitric oxide in the body. The vasodilating antihypertensive drug minoxidil contains an ·NO moiety and may act as an NO agonist.

Exemplary nitric oxide donors for use in the present invention include, without limitation, isosorbide dinitrate, L-arginine, linsidomine, minoxidil, nicorandil, nitroglycerin, nitroprusside, nitrosoglutathione, and S-nitroso-N-acetyl-penicillamine (SNAP), sodium nitroprusside (SNP also known as Nitropress), glyceryl trinitrate (GTN), isosorbide mononitrate (ISMN), pentaerythrityl tetranitratethe (PETN), ‘NONOates’ (e.g diazeniumdiolates), S-nitrosothiol class (e.g. GSNO).

Accordingly in a particular embodiment, the NO donor is selected from the list consisting of isosorbide dinitrate, L-arginine, linsidomine, minoxidil, nicorandil, nitroglycerin, nitroprusside, nitrosoglutathione, and S-nitroso-N-acetyl-penicillamine (SNAP), sodium nitroprusside (SNP also known as Nitropress), glyceryl trinitrate (GTN), isosorbide mononitrate (ISMN), pentaerythrityl tetranitratethe (PETN), ‘NONOates’ (e.g diazeniumdiolates), S-nitrosothiol class (e.g. GSNO).

In a particular embodiment, linsidomine or minoxidil or SNAP or SNP are the preferred agent.

In an additional aspect the invention relates to a method of preventing or treating sexual dysfunction associated with a Polycystic Ovary Syndrome (PCOS) in a patient in need thereof comprising administering to the patient a therapeutically effective amount of Nitric Oxide (NO) agent.

In its broadest meaning, the term “treating” or “treatment” refers to reversing, alleviating, inhibiting the progress of neurological symptoms especially sexual dysfunctions associated with Polycystic Ovary Syndrome (PCOS) patient. In particular, “prevention” or “prophylactic treatment” of sexual dysfunctions associated with Polycystic Ovary Syndrome (PCOS) may refer to the administration of the compounds of the present invention that prevent the neurological symptoms especially sexual dysfunctions associated with of Polycystic Ovary Syndrome (PCOS).

The inventors demonstrated that the loss of NO expression in the hypothalamic VMH, ARC and R3PV is associated with altered sexual behavior and that pharmacological replenishment of NO restores normal sexual behavior in PCOS-like mice. Moreover, the inventors showed that SNAP treatment restores normal neuronal activity (cFOS expression) in many hypothalamic, limbic and mesolimbic circuits associated with both appetitive and consummatory sexual behavior. These data suggest that impaired brain NO-signalling may also be present in other forms of altered sexual desire in humans.

Accordingly in an additional aspect the invention relates to a method of preventing or treating of sexual dysfunction in a patient in need thereof comprising administering to the patient a therapeutically effective amount of Nitric Oxide (NO).

In a particular embodiment the sexual dysfunction is associated with hypoactive sexual desire disorder (HSDD).

The term “Hypoactive sexual desire disorder” (HSDD) or hyposexuality or inhibited sexual desire (ISD) “has its general meaning in the art refers to the most severe form of low sexual drive and is considered a sexual dysfunction in some jurisdictions and is characterized as a lack or absence of sexual fantasies and desire for sexual activity, as judged by a clinician. For this to be regarded as a disorder, it must cause marked distress or interpersonal difficulties and not be better accounted for by another mental disorder, a drug (legal or illegal), or some other medical condition. A person with ISD will not start, or respond to their partner's desire for, sexual activity. HSDD affects approximately 10% of all pre-menopausal women in the United States, or about 6 million women.

There are various subtypes. HSDD can be general (general lack of sexual desire) or situational (still has sexual desire, but lacks sexual desire for current partner), and it can be acquired (HSDD started after a period of normal sexual functioning) or lifelong (the person has always had no/low sexual desire.)

In the DSM-5 (The Diagnostic and Statistical Manual of Mental Disorders, Fifth Edition, is the 2013 update to the Diagnostic and Statistical Manual of Mental Disorders, the taxonomic and diagnostic tool published by the American Psychiatric Association (APA)), HSDD was split into male hypoactive sexual desire disorder and female sexual interest/arousal disorder. Other terms used to describe the phenomenon include sexual aversion and sexual apathy. More informal or colloquial terms are frigidity and frigidness.

Typically, medicaments according to the invention comprise a pharmaceutically-acceptable carrier. A person skilled in the art will be aware of suitable carriers. Suitable formulations for administration by any desired route may be prepared by standard methods, for example by reference to well-known text such as Remington; The Science and Practice of Pharmacy.

### Pharmaceutical Composition and Kit

The compounds of the invention (NO agent: inhaled Nitric Oxyde (iNO) or NO donor) may be used or prepared in a pharmaceutical composition.

In one embodiment, the invention relates to a pharmaceutical composition comprising the compound of the invention and a pharmaceutical acceptable carrier for use in the treatment or prevention of sexual dysfunction associated with a Polycystic Ovary Syndrome (PCOS) in a subject of need thereof.

Typically, the compound of the invention may be combined with pharmaceutically acceptable excipients, and optionally sustained-release matrices, such as biodegradable polymers, to form therapeutic compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intra-peritoneal, intramuscular, intravenous, sub-dermal, transdermal, intrathecal and intranasal administration forms and rectal administration forms.

Preferably, the pharmaceutical compositions contain vehicles, which are pharmaceutically acceptable for a formulation capable of being administered by oral-route forms for NO donor (see the above list) or by intranasal or intrapulmonary route form for inhaled NO (iNO).

In addition to the compounds of the invention formulated for oral administration, other pharmaceutically acceptable forms include, e.g. tablets or other solids for oral administration; liposomal formulations; time-release capsules; and any other form currently used.

Vehicles used usually for iNO (inhaled Nitric Oxide) may contain carrier gas such as nitrogen or helium.

The invention also provides kits comprising the compound of the invention. Kits containing the compound of the invention find use in therapeutic methods.

The invention will be further illustrated by the following figures and examples. However, these examples and figures should not be interpreted in any way as limiting the scope of the present invention.

### FIGURES

FIG. 1: PAMH female mice display sexual dysfunction during adulthood. (A) Scheme shows how PAMH female mice are generated following AMH excess over late pregnancy. Behavioral assessment for female sexual behavior was performed with adult PAMH female mice. (B) Histogram shows lordosis quotient (LQ) in control (N=7) and PAMH (N=7) female mice depicting a significant impairment of the sexual behavior in a PCOS-like condition. Unpaired Student's t-test; *** P<0.001. (C) Number of male mounting trials over the 15-min lordosis test. Males were paired with control (N=7) and PAMH (N=7) female mice. Unpaired Student t-test; * P<0.05. (D) Histogram shows LQ from paired intact and ovariectomized female mice with estradiol (E2)/progesterone (P4) supplementation (E2+P4) in control (N=7) and PAMH (N=7) groups. Two-way ANOVA with Tukey's post hoc test. (E) Representative drawings showing typical sexual rejection behaviors in female mice, which are categorized as mild rejection (arching and escaping) and severe rejection (kicking and boxing) behavior. (F-G) Mild rejection quotient (MRQ) and severe rejection quotient (SRQ) in paired intact and ovariectomized female mice treated with E2+P4 in control (N=7) and PAMH (N=7) groups. Two-way ANOVA with Tukey's post hoc test. (H) Representative drawing showing how mate preference test is performed in control and PAMH female mice (see more details in methods). (I) Histogram representation of the time spent by control (N=8) and PAMH (N=8) female mice exploring the different compartments of the mate preference test box. Each phenotype was analyzed separately. Two-way ANOVA with Tukey's post hoc test. (J) Preference score for mate preference test in control (N=8) and PAMH (N=8) female mice. The blue bar indicates male-directed preference while the pink bar indicates female-directed preference. H0: mean equals 0; unpaired Student's t-test; ** P<0.01. Data are represented as mean±SEM. Different letters indicate statistically significant differences.

FIG. 2: Sexual dysfunction in PAMH mice is associated with decreased expression of neuronal nitric oxide synthase (nNOS) and progesterone receptor (PR) in the hypothalamus. (A-C) Number of nNOS-, PR-, and nNOS+PR-expressing cells in the RP3V in control (N=6) and PAMH (N=5) female mice. Unpaired Student's t test; **** P<0.0001. (D) Percentage of nNOS neurons co-expressing PR in the RP3V in control (N=6) and PAMH (N=5) female mice. Mann-Whitney U test. (E-G) Number of nNOS-, PR-, and nNOS+PR-expressing cells in the VMH in control (N=8) and PAMH (N=7) female mice. Unpaired Student's t test; ** P<0.01; *** P<0.001; **** P<0.0001. (H) Percentage of nNOS neurons co-expressing PR in the VMH in control (N=8) and PAMH (N=7) female mice. Mann-Whitney U test. (I-K) Number of nNOS-, PR-, and nNOS+PR-expressing cells in the RP3V in control (N=8) and PAMH (N=7) female mice. Unpaired Student's t test; * P<0.05; *** P<0.001. (L) Percentage of nNOS neurons co-expressing PR in the ARN in control (N=8) and PAMH (N=7) female mice. Mann-Whitney U test. Data are represented as mean±SEM.

FIG. 3: Functional requirement of NOergic signaling and nNOSVMH neurons for the control of female sexual behavior. (A-C) Lordosis, mild rejection, and severe rejection quotients following the administration of 8 mg/kg S-nitroso-N-Acetyl-DL-Penicillamine (SNAP; s.c. injection) 15 minutes before the behavioral test in control (N=11) and PAMH (N=8) female mice. Two-way ANOVA with Tukey's post hoc test. (D-F) Lordosis, mild rejection, and severe rejection quotients following the administration of 15 mg/kg sildenafil citrate (i.p. injection) one hour before the behavioral test in control (N=6) and PAMH (N=10) female mice. Two-way ANOVA with Tukey's post hoc test . . . ((G-I) Lordosis, mild rejection and severe rejection quotients following chemogenetic inhibition of nNOSVMH neurons in female mice. Acute chemogenetic manipulation with 3 mg/kg CNO promotes sexual dysfunction and enhanced sexual rejection in Nos1cre/+ female mice (N=10). Wilcoxon signed-rank test; ** P<0.01. Data are represented as mean±SEM.

FIG. 4: Kisspeptin does not rescue normal sexual behavior following acute inhibition of nNOS^{VMH} neurons. To study the functional coupling of kisspeptin-nNOS^{VMH} neurons, both wild-type (WT) control and Nos1^{cre/+} female mice were subjected to a pre-treatment with 3 mg/kg CNO (i.p.) and treatment with kisspeptin-10 (Kiss-10) before lordosis test. (A) Evaluation of lordosis behavior in experienced WT (N=7) and Nos1^{cre/+} (N=6) female mice following Cre-dependent chemogenetic inhibition of nNOS^{VMH} neurons. Comparison analysis was performed within each group separately. Repeated-measures one-way ANOVA with Tukey's post hoc test; * P<0.05; ** P<0.01. ((B-C) Number of GFP immunoreactivity and co-expression between GFP and PR within Kiss1^{RP3V} neurons in control (N=6) and PAMH (N=5) female mice. Unpaired Student's/test; *** P<0.001; **** P<0.0001. (D) Percentage of colocalization between GFP and PR within Kiss1RP3V neurons in control (N=6) and PAMH (N=5) female mice. Mann-Whitney (/test. Data are represented as mean±SEM.

FIG. 5. Proposed AMH-mediated developmental drive of brain circuit abnormalities leading to the manifestation of sexual dysfunction in PCOS. (A) Excessive AMH production by PCOS ovaries access the mother's brain leading to neuroendocrine dysfunction promoting high LH secretion. Exacerbated LH signaling in the ovaries disrupts hormonal homeostasis favoring enhanced ovarian testosterone (T) synthesis and high T levels access the developing female fetal brain. AMH also deregulates placental steroidogenesis, which increases T bioavailability as previously demonstrated by our group (30). (B) Prenatal AMH and testosterone excess reprogramming might mediate the loss of progesterone (P4) sensitive nNOS neurons in different hypothalamic nuclei involved in reproductive (56) and sexual competence. These brain circuit abnormalities might be present already during prepubertal life in PCOS girls (29, 78) and are carried over reproductive maturation. (C) Following the developmental reprogramming in hypothalamic circuitry, such as nNOS pathways, PCOS women might be more likely to manifest both neuroendocrine and sexual dysfunction during adult reproductive life.

### EXAMPLE

### Materials and Methods

Animals. Mice were housed under specific pathogen-free conditions in a temperature-controlled room (21-22° C.) and ad libitum access to food and drinking water. Unless stated in the text, mice were kept under an inverted 12-h light: dark cycle with lights off from 9:00 h to 21:00 h. All experiments were performed using adult (2-5 months of age) mice. C57BL/6J mice were purchased from Charles River Laboratories (USA). Nos1^{cre/+} (Nos1-ires-cre; B6.129-Nos1^{tm1(cre)Mgmj}/J) female mice were purchased from Jackson Laboratory (USA; JAX stock #017526) (51). Kiss1-GFP (kisspeptin-IRES-Cre; KissIC) (52) were donated by Professor Ulrich Boehm (Saarland University, Germany). Animal care and experimental design were carried out under the guidelines established by the European Council Directive of 22 Sep. 2010 (2010/63/EU) and under ethical protocol number APAFIS #29172-2020121811279767 v5 from the Institutional Ethics Committees of Care and Use of Experimental Animals of the University of Lille, France.

Prenatal anti-Müllerian hormone (PAMH) treatment. The generation of PAMH female mice was carried out according to previous report (30).

Assessment of reproductive status, LH pulse profiling, and blood sampling. Estrous cycle pattern and length were assessed in adult control and PAMH mice as stated elsewhere (71, 72).

Hormone measurements and analysis. Blood sampling for both LH and testosterone measurements was collected over the dark phase 3h after lights were turned off. Blood LH levels were determined using a well-established ELISA method (72) (SI Appendix). Plasma testosterone levels were measured in duplicates using a commercial ELISA kit (Demeditec Diagnostics, GmbH, DEV9911) according to the manufacturers' instructions. The assay sensitivity of this mouse testosterone ELISA was 0.066 ng/ml, and the intra-assay coefficient of variation was 10.4%.

Immunohistochemistry. Tissue preparation for immunohistochemistry is shown in SI Appendix. Free-floating immunohistochemistry was performed as described elsewhere (29, 73). Primary antibodies were used at the respective concentrations: polyclonal sheep anti-nNOS (1:2,000; generous gift from Dr. Piers C. Emson and validated elsewhere (74)), polyclonal rabbit anti-PR (1:500; Sigma; catalog #SAB4502184), rabbit anti-red fluorescent protein (RPF) (1:500; Rockland Immunochemicals, Inc.; catalog #600-401-379), and polyclonal chicken anti-GFP (1:1,000; Aves Labs Inc., catalog #GFP-1020). Brain sections were incubated with a cocktail of primary antibodies and 2% normal donkey serum (NDS) in incubation solution (0.25% bovine serum albumin with 0.3% Triton in TBS) over 48 hours at 4° C. Following washes with TBS, brain sections were incubated with secondary antibodies. Alexa Fluor 488 donkey anti-sheep (reference #A11015), Alexa Fluor 647 donkey anti-rabbit, (reference #A31573), Alexa Fluor 568 donkey anti-rabbit (reference #A10042) were all purchased from Thermo Fisher Scientific Inc. and used at 1:400 dilution in TBS solution. Alexa Fluor 488 donkey anti-chicken (Jackson ImmunoResearch Labs; reference #703-545-155) was used at 1:500 dilution in TBS solution. Sections were mounted onto glass slides and left to dry until covered with Fluoromount-G™ with DAPI (Invitrogen/Thermo Fisher Scientific Inc.; reference #00-4959-52), and coverslipped.

Image acquisition and analysis. Two representative brain sections from each region (RP3V, VMH, rARN, mARN, and cARN) were chosen from each animal for Immunohistological analysis. Brain sections were firstly examined using an Axio Imager, Z1 ApoTome microscope (Zeiss, Oberkochen, Germany), and an AxioCam MRm camera (Zeiss). Confocal imaging and analyses Zeiss Spinning-Disk microscope Yokogawa CSU-X1 model (Zeiss; BioImaging Center Lille (BICeL), of the University of Lille, France). High magnification images were acquired taking Z-stacks of 0.5 μm-steps using a 40x-oil objective (1.30 NA). Confocal images were further processed with ImageJ software for cell counting analysis. Final analysis considered the average counting from two representative sections of each region.

Ovariectomy and ovarian steroid hormone supplementation. Adult female mice (3-months old) were ovariectomized under 4% isoflurane anesthesia with constant airflow. Two weeks following surgery and recovery female mice were primed with E2+P4 to have high sexual receptivity based on previous reports (75). To this end, mice received s.c. injections of 0.5 μg/50 μL estradiol benzoate (Sigma-Aldrich; reference #E8515) 48h and 24h prior lordosis test, and a single s.c. injection of 500 μg/50 μL progesterone (Sigma-Aldrich; reference #P0130) 3-4 h before lordosis test. Both drugs were diluted in sesame oil at least one day before the protocol commenced.

Female sexual behavior test. Estrus female mice were tested for lordosis behavior over the dark phase following previously reported protocols (25, 40). During the annotation of sexual behavior, we also assessed sexual rejection behaviors over the same period of time.

All behavior tests were videotaped and manually annotated using Behavioral Observation Research Interactive Software (BORIS) (76).

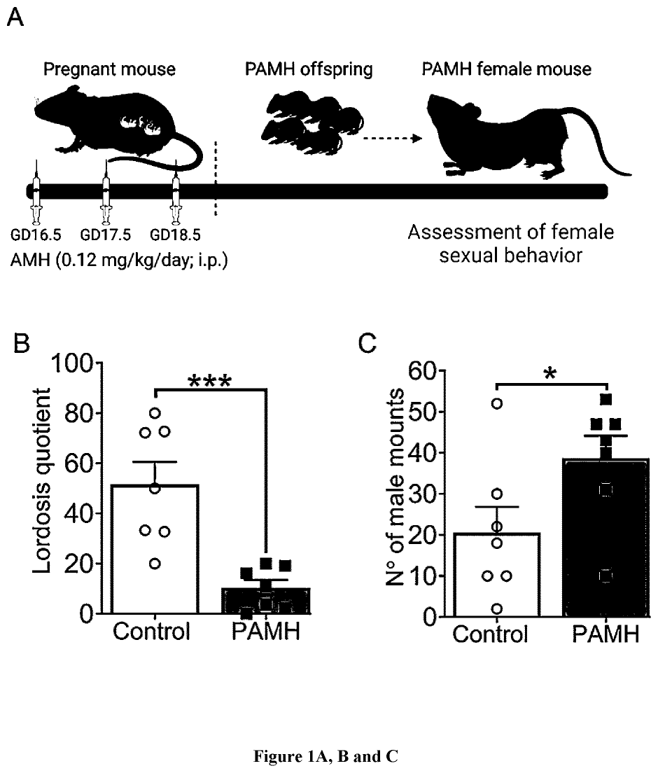

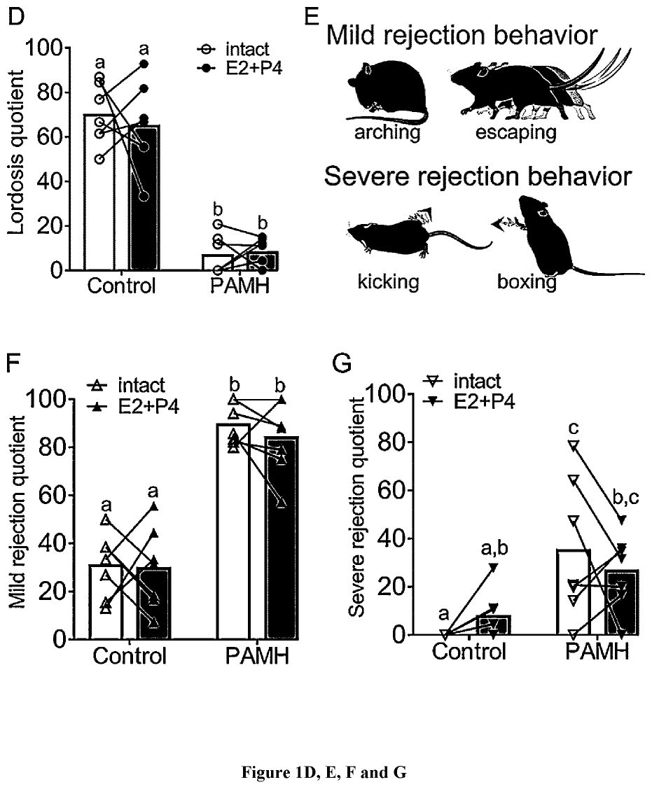

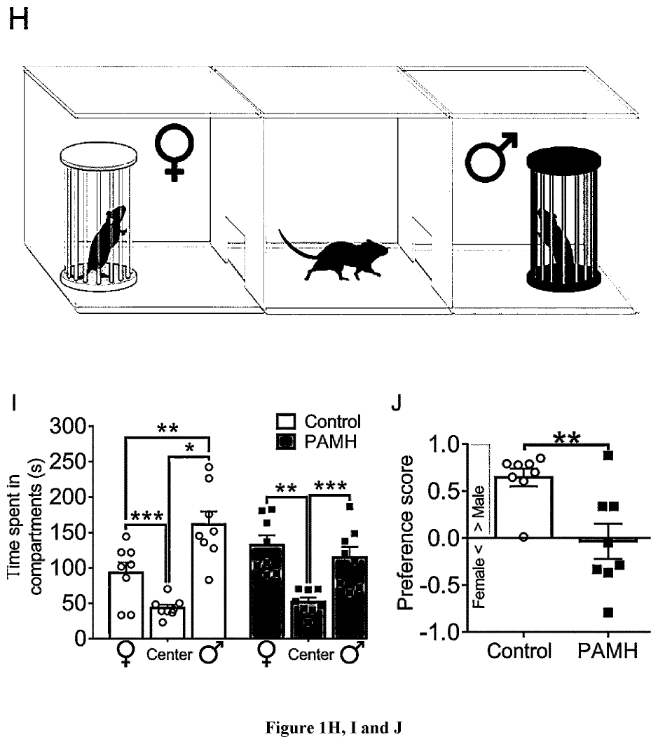

Mate preference test. The mate preference test assessed female mice's response sensory stimuli to same and opposite-sex counterparts using a three-compartment box as shown in FIG. 1. Technical details are described in SI Appendix. The exploration time spent exploring each chamber was determined and the preference score was calculated as the ratio of time exploring the male chamber minus the time exploring the female chamber divided by the total time of exploring both opposite chambers.

Elevated plus maze (EPM) test. The EPM test was employed as previously reported elsewhere (39) and details are provided in SI Appendix.

Chemogenetic manipulations (viral transfection, stereotaxic injections, and behavioral assessment). The AAV9 double floxed Gi-coupled hM4D DREADD fused with fluorescent mCherry protein under the control of human synapsin promoter (AAV-hSyn-DIO-hM4D(Gi)-mCherry) (Addgene; catalog #44362-AAV9) was stereotaxically injected into the vlVMH of Nosl-ires-cre female mice under 4% isoflurane anesthesia with constant airflow. Lordosis test was carried out as described above with sexually experience female mice after three lordosis test trials. Females received i.p. injection with 100 μL of clozapine N-oxide dihydrochloride (CNO; Tocris; catalog #6329), at a dose of 3 mg/kg diluted in sterile saline, one hour before behavioral assessment. Each mouse received either CNO or saline (vehicle) in a random order, and the two behavioral assessments were performed ten days apart.

Pharmacological treatments. SNAP: Female mice received a subcutaneous (s.c.) injection with 8 mg/kg S-nitroso-N-Acetyl-DL-Penicillamine (SNAP) (Sigma-Aldrich; catalog ##N3398) diluted in 100 μL of saline 15 minutes before behavioral testing and the chosen dose was based on previous reports (25). Kisspeptin: Female mice received a s.c. injection with 0.52 μg/kg kisspeptin-10 (Kiss-10; rodent metastin (45-54) amide; GeneCust; YY-10-NH2) diluted in 100 μL of saline 30 minutes before behavioral test based on previous reports (25). Sildenafil citrate: Female mice received an i.p. injection with 15 mg/kg sildenafil citrate (Sigma-Aldrich; catalog #171599-83-0) 1h before behavioral assessment based on previous trials from our laboratory (Chachlaki et al., unpublished data). Sildenafil citrate was firstly diluted in dimethyl sulfoxide (DMSO) to make up a stock solution of 25 mg/mL and further diluted with sterile saline to make up a working solution of 15 mg/mL for s.c. injection on the day of the injection.

Data analysis and statistics. Statistical analysis was performed with PRISM software 8.0 (GraphPad Software, San Diego, CA, USA). Normal distribution was determined with the Shapiro-Wilk normality test for all samples before any group analysis. Sample sizes were chosen according to standard practices and are shown in each figure legend. Investigators were not blinded to the group allocation or drug treatment; however, each mouse was randomly chosen to receive different treatments on different days. We used the Mann-Whitney (/test to compare two experimental groups in which unpaired samples were not normally distributed. For normally distributed unpaired samples, we used a two-tailed Student's t-test. Paired samples were analyzed using Wilcoxon signed-rank test (e.g., chemogenetic manipulations) or repeated-measures one-way ANOVA (e.g., chemogenetic manipulations with Kiss-10 pre-treatment). Group analysis with two nominal predictor variables (e.g., phenotype and treatment) used two-way ANOVA with either Tukey's or Sidak's post hoc test. Statistical significance was accepted when P<0.05. Additional information is displayed in figure legends of their respective graphical analysis.

### Results

### PCOS-Like Animals Show Altered Sexual Behavior.

To date, alterations in female sexual behavior in preclinical models of PCOS are not well-described. To fill this knowledge gap, we generated prenatally-treated AMH (PAMH) female mice (30) and performed a thorough neuroanatomical and behavioral assessment of these animals (FIG. 1A). We confirmed that adult PAMH female mice present substantial disruption of reproductive cycles, wherein PAMH animals spend less time in proestrus, the preovulatory stage (P<0.0001), and more time in metestrus (P<0.05) than control females (Data not shown). Hyperandrogenemia is a hallmark of PCOS (37) and preclinical PCOS models (34); however, circulating testosterone levels are commonly measured over the light phase of light: dark cycles. We assessed plasma testosterone levels on the day of estrus (receptive phase) over the dark phase when naturally nocturnal mice display full sexual behavior. We found that PAMH female mice have higher plasma testosterone levels (˜ 8.62-fold increase) than control female mice (Data not shown; P<0.0001). Neuroendocrine disruptions contribute to increased testosterone production through high luteinizing hormone (LH) pulse secretion and, hence, LH actions in PCOS ovaries (29, 35). We found that diestrous PAMH mice exhibit high pulsatile LH secretion over the dark phase of the light: dark cycles (Data not shown). Specifically, PAMH females exhibit higher mean LH levels (Data not shown; P<0.01), pulse frequency of LH release (FIG. S1H; P<0.0001), and integrated LH response (FIG. 1SI; area under the curve analysis; P<0.01) than control female mice.

Copulatory behavior in female rodents is typically displayed as lordosis over the consummatory phase being primarily dependent upon the proper functioning of brain circuitry. Thus, female mice were tested for lordosis behavior to determine whether the prenatal AMH insult also promotes the disruption of sexual receptivity and performance in adulthood (FIG. 1). PAMH female mice displayed significantly fewer lordosis responses compared with control females (FIG. 1B; P<0.001; lordosis quotient (LQ); LQControl=51.57±8.96 vs. LQPAMH=10.33±3.17). This disruption in sexual behavior was not attributed to a loss of mating partner's interest as male mice attempted to mount PAMH female mice more often than control females (FIG. 1C; P<0.05). The latency for the first mount was not different between the groups taking on average 309.4±148.2 s for the first mounting attempt in males paired with control females versus 408.8±110.2 s for males paired with PAMH female mice (Mann-Whitney U test; P=0.58).

Decreased sexual motivation and performance are frequently associated with high levels of anxiety in both healthy and PCOS women (38). Thus, we tested whether PAMH treatment could also promote anxiety-like behavior as a primary culprit toward sexual dysfunction employing the elevated plus maze (EPM) test, in which spending less time in open areas of the maze as a valid index of anxiety-like behavior (39). Analysis indicated that prenatal AMH treatment does not affect the time spent in open areas such as the open arm and central part of the maze (Data not shown). We observed a mild increase in the time spent in the closed arms of the maze by PAMH females compared with controls (Data not shown; P<0.05). Increased time in closed arms could indicate normal proclivity toward dark enclosed areas following risk assessment and avoidance of open spaces. However, we also did not detect any differences in the number of entries in either closed or open arms (Data not shown). The ratio of time spent in the open arms compared to the other areas of the maze (Data not shown) and the latency to enter open arms (Data not shown) were also similar between the two groups. Together, our findings reveal that PAMH female mice display a pronounced impairment of the sexual performance, which is unlikely to be related an anxiety-like condition.

### Sexual Dysfunction and Sexual Rejection Behaviors are not Dependent Upon Circulating Ovarian Hormone Levels in PCOS-Like Mice.

Ovarian steroid hormone actions in the female brain by estradiol (E2) and progesterone (P4) are necessary to increase sexual motivation and the display of adequate lordosis behavior (40), respectively. Thus, differences in endogenous E2 and P4 levels could influence lordosis behavior in PAMH female mice. To investigate this possibility, we assessed lordosis behavior in control and PAMH female mice before (intact) and after ovariectomy followed by E2 and P4 supplementation. We found that E2+P4 treatment does not affect lordosis behavior in both groups, and that PAMH female mice still display impaired sexual behavior in comparison with control females (FIG. 1D; effect of the E2+P4 treatment: P=0.73, F1,24=0.12; effect of the phenotype: P<0.0001, F1,24=148.00). These results suggest that intrinsic defects may arise from developmental disruptions in the PCOS-like condition most likely impinging on the E2/P4 signaling pathways and that sex hormone replenishment in adulthood is not sufficient to recover normal sexual performance.

Social interactions between female and male mice toward copulation are facilitated when females are highly receptive. Conversely, during unreceptive states, females may display rejecting behaviors in response to male mounting attempts. Based on previous reports (41, 42), we categorized these sexual rejection behaviors into two types: mild rejection, in which female mice display either arching the back to prevent mounting or escaping; and severe rejection when females engage with hindlimb kicking and boxing following mounting attempts (FIG. 1E). We observed that while intact control female mice in estrus display a low mild rejection quotient (MRQ=30.2) and severe rejection quotient (SRQ=3.8), intact PAMH female mice in estrus spend more time rejecting male mounts with high rates of both mild (MRQ=86.7) and severe (SRQ=30.8) rejecting behaviors (FIGS. 1F and 1G; MRQ control vs. PAMH=P<0.0001; SRQ control vs. PAMH=P<0.01). We also observed that hormone supplementation did not change the magnitude of these behaviors in both groups for either mild (effect of the E2+P4 treatment: P=0.53, F1,24=0.39) or severe rejection (effect of the E2+P4 treatment: P=0.96, F1,24=0.003) in comparison when the same animals were firstly tested in intact estrus (FIGS. 1F and 1G).

As PAMH mice present a robust disruption of sexual performance and enhanced display of rejecting behaviors, we also investigated whether the mate preference was altered in these animals. We assessed mate preference using a two-chamber test in which one side contained an experienced and receptive female mouse and the opposite side contained an experienced male mouse (FIG. 1H). Group analysis revealed that control female mice spend more time exploring the chamber with an experienced male (P<0.01), whereas PAMH female mice spend similar time exploring both chambers (FIG. 1J). Further analysis confirmed that, unlike control females, which present a typical male-directed preference, PAMH female mice failed to show any sex-directed preference (FIG. 1I; unpaired Student's t-test; H0: mean equals 0; control vs. PAMH=P<0.01). This loss of male-directed preference may contribute to the impairment in sexual function observed in PAMH female mice.

Sexual Dysfunction in PAMH Mice is Associated with Decreased Expression of Neuronal Nitric Oxide Synthase (nNOS) and Progesterone Receptor (PR) in the Hypothalamus.

Among multiple signal transduction pathways governing female sexual behavior, both NOergic signaling and progesterone actions are well-known to trigger female lordosis (23, 40, 43, 44). Women with PCOS have both low circulating levels of NO metabolites (45, 46) and impaired progesterone-mediated negative feedback (35, 47). Therefore, we hypothesized that the expression of nNOS and PR could be disrupted in PAMH female mice as they exhibit both neuroendocrine and sexual dysfunction. We mapped the expression of nNOS and PR in different hypothalamic regions known to be involved in female sexual behavior (17, 22, 25, 48): rostral periventricular area of the third ventricle (RP3V), ventromedial nucleus of the hypothalamus (VMH), and arcuate nucleus (ARN) during estrus.

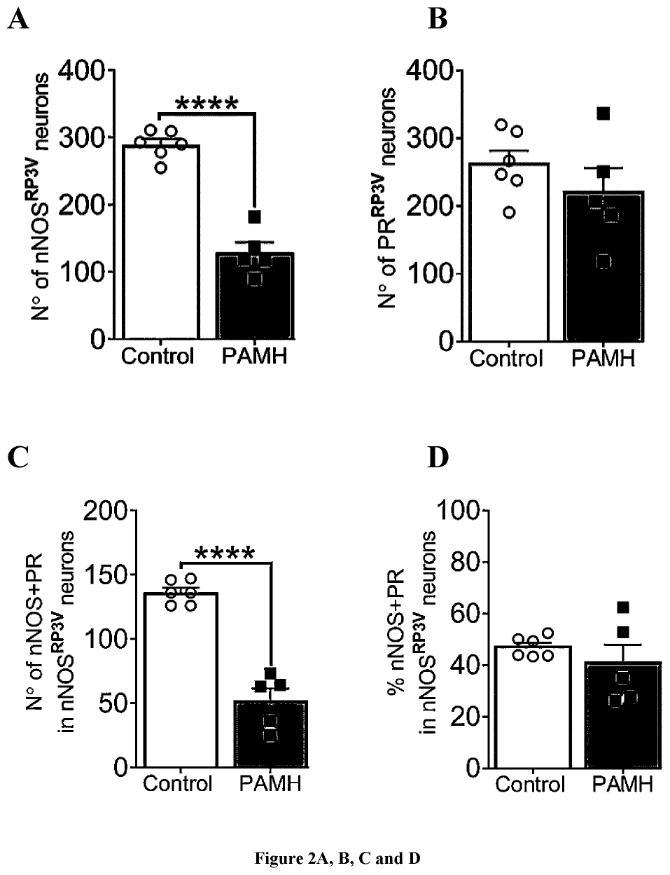

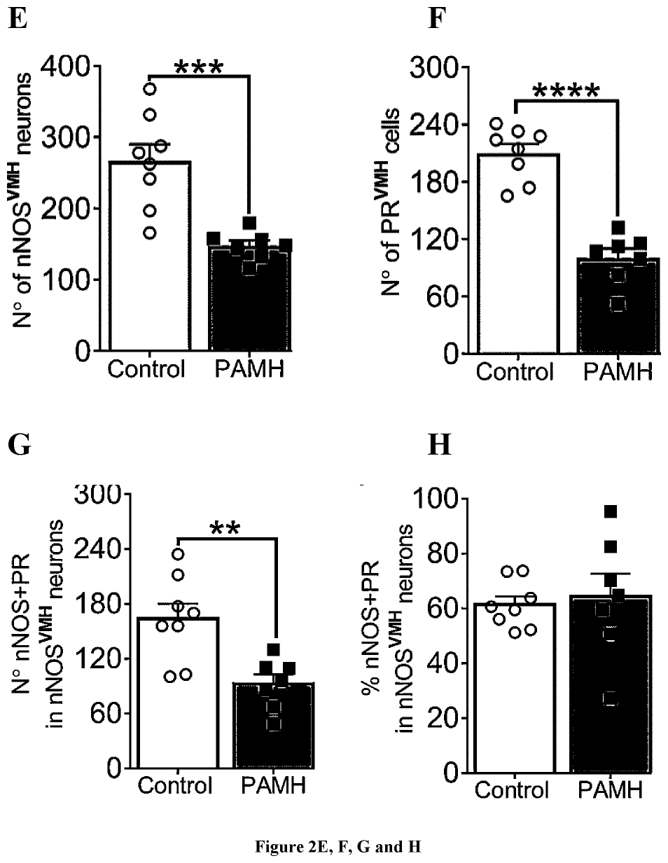

Firstly, we documented by immunohistochemical studies a robust 50% reduction in the number of nNOS neurons located in the RP3V (nNOSRP3V; Data not shown) in PAMH female mice compared with control females (FIG. 2A; P<0.0001). The expression of PR in the RP3V (PRRP3V) was not altered by PAMH treatment (FIG. 2B), whereas the number of nNOSRP3V neurons co-expressing PR was significantly lower in PAMH mice than controls (FIG. 2C; 61.7% lower in PAMH; P<0.0001). The percentage of colocalization between nNOSRP3V and PRRP3V was similar in both groups (FIG. 2D; P=0.66; control=47.22±1.5% vs. PAMH=40.91±7.2%).

Next, we investigated the expression of both proteins in the VMH, which is highly important for the progesterone-mediated sexual performance in females (20, 22) and the integration of neuroendocrine functions (Data not shown). We discovered that PAMH female mice exhibit a 44.5% less nNOS neurons in the VMH (nNOS^{VMH}) when compared with controls (FIG. 2E; P<0.001). A similar shift was also observed for both cells expressing PR (PRVMH) (FIG. 2N; 52.2% reduction; P<0.0001) and nNOS^{VMH} neurons co-expressing PR (FIG. 2G; 43.4% reduction; P<0.01) in PAMH female mice compared with control females. Although these protein expressions were attenuated in PAMH mice, the percentage of nNOS^{VMH} expressing PRVMH colocalization was similar between the groups (FIG. 2H; control=61.3% vs. PAMH=64.3%; P=0.69) indicating that the reduction of the number of PRVMH cells may be dependent upon the reduction of nNOS^{VMH} expression.

We then investigated whether the expression of nNOS and PR in the ARN (nNOS^{ARN} and PR^{ARN}, respectively) (Data not shown) could be disturbed in a PCOS-like condition. Immunohistochemical analysis showed that the number of nNOS^{ARN} neurons (FIG. 2I; P<0.001), PRARN cells (FIG. 2J; P<0.001), and nNOS^{ARN} neurons co-expressing PR (FIG. 2J; P<0.05) were significantly lower in PAMH than controls. The ARN is an elongated region stretching caudally throughout the medial basal hypothalamus (MBH); thus, we carried out a refined analysis of different parts of the ARN to identify specific regions with abnormal nNOS and PR expression. The lower number of nNOS^{ARN} neurons in PAMH female mice was due to a robust decrease in the middle part of the nucleus (mARN; bregma=−1.55 mm↔−2.03 mm; P<0.01), whereas the rostral (rARN; bregma=−1.23 mm↔−2.03 mm) and caudal part (rARN; bregma=−2.03 mm↔−2.53 mm) did not seem to be significantly affected by PAMH treatment (Data not shown). Oppositely, PR expression was decreased along with the entire extension of the ARN in PAMH females (Data not shown). The co-expression of PR and nNOS was the lowest among the three hypothalamic regions for both groups (FIG. 2L; control=10.5±2.3% vs. PAMH=7.1±2.0%; P=0.42) and the number of nNOS^{ARN} neurons co-expressing PR was only significantly decreased in the mARN region (Data not shown).

We further analyzed the percentage of PR cells co-expressing nNOS in the three hypothalamic nuclei. We found that within each nucleus, control and PAMH female mice express similar percentages of co-expression of the two proteins in PR cells (Data not shown). Among the three nuclei, the VMH contained the highest colocalization rate with 91.6±2.5% and 92.1±3.6 of PR cells co-expressing nNOS in control and PAMH mice, respectively (Data not shown; effect of hypothalamic nuclei=F2, 30=216.7; P<0.0001). PR^{RP3V} cells co-expressed 43.3±6.9% and 32.12±6.9% in control and PAMH mice, respectively, whereas the ARN displayed the lowest colocalization rate with 5.6±0.9% and 5.0±1.2±in control and PAMH mice, respectively (Data not shown).

As gonadal hormones influence the expression of nNOS in hypothalamic and limbic regions in both sexes in different mammalian species (49, 50), we further examined whether the hyperandrogenism of PAMH animals could negatively impact nNOS expression in other brain regions involved in the control of sexual behavior (30, 51-53). Histological analysis from brain sections containing the medial preoptic area (MPA), median preoptic nucleus (MnPO), anterior division of the bed nucleus of the stria terminalis (aBNST), and posterodorsal medial amygdala nucleus (pdMeA) did not reveal any difference in the number of nNOS neurons in these regions between control and PAMH female mice (Table 1).

Taken together, these data illustrate a robust association between neuroendocrine and sexual behavior impairments with a disruption of nNOS and PR expression in specific regions of the hypothalamus of PCOS-like female mice.

### Peripheral Injection of NO Donor Restores Normal Sexual Behavior and Attenuates Sexual Rejection in PAMH Mice.

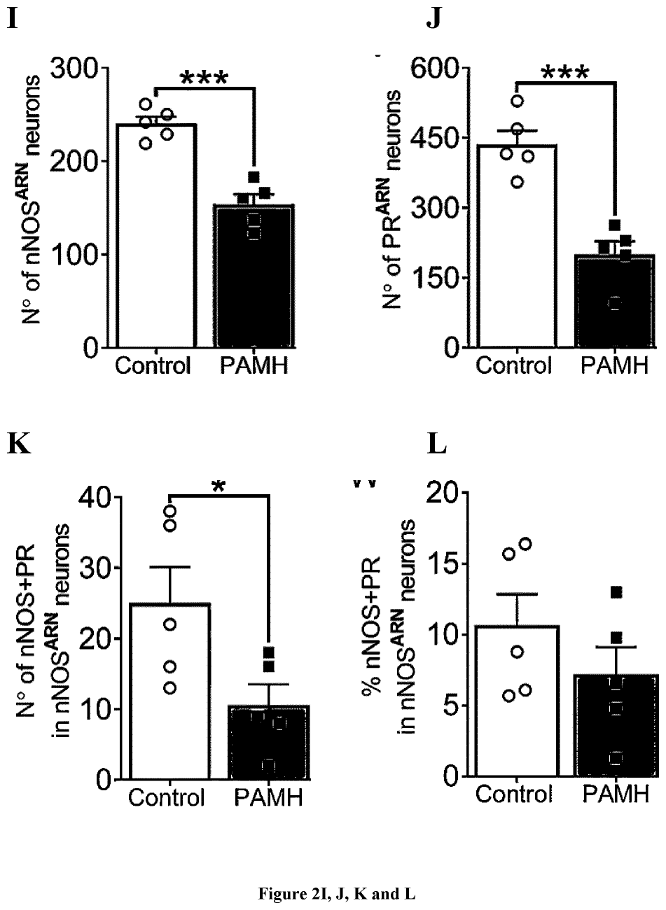

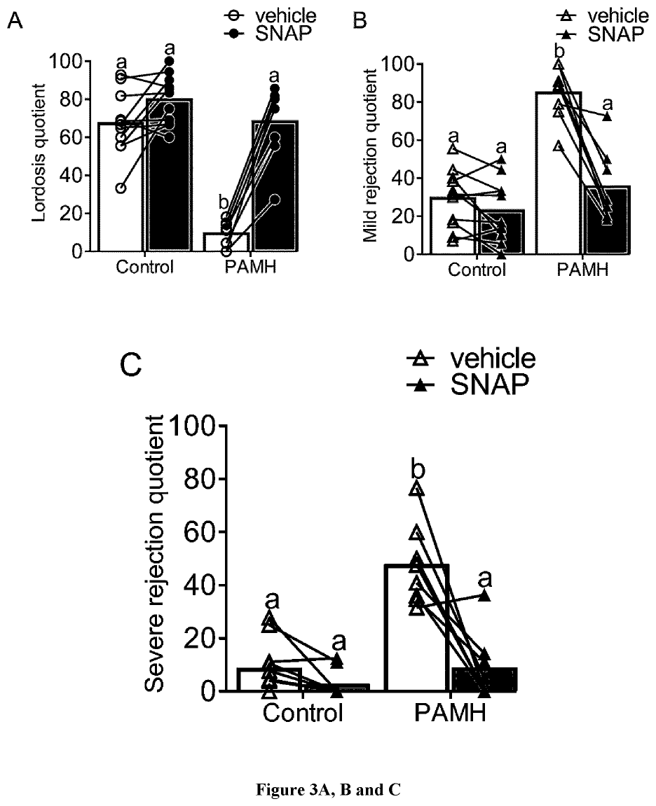

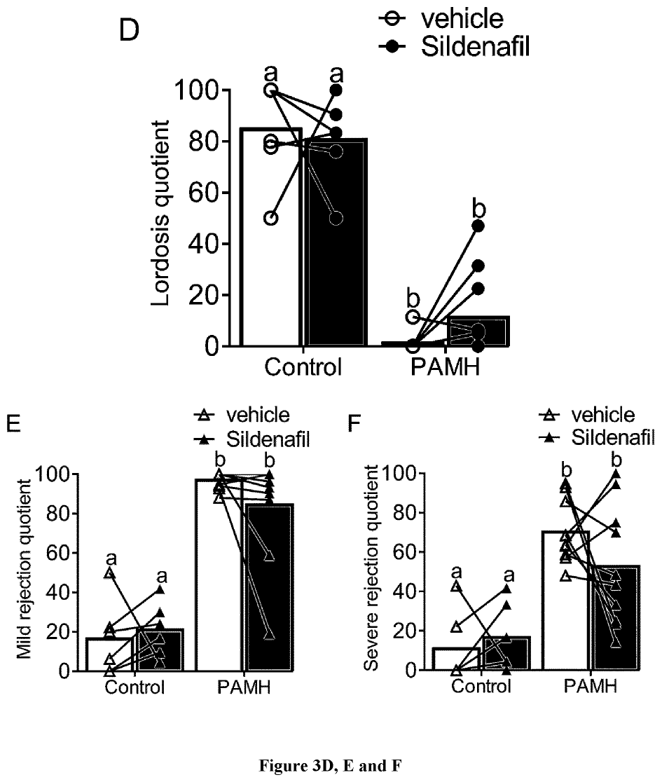

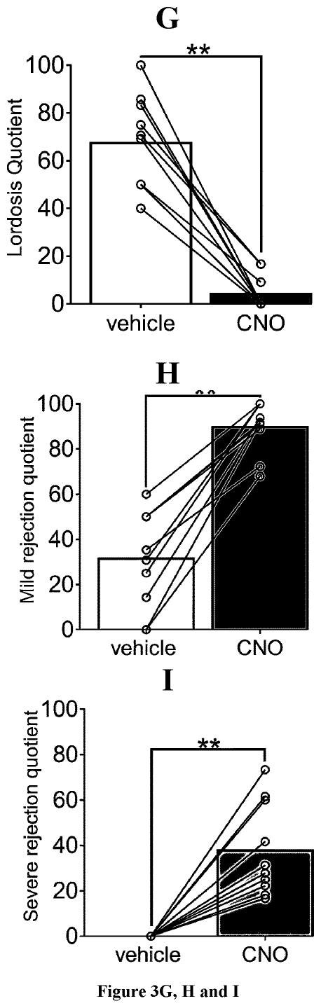

Previous reports indicate that the control of female sexual behavior is highly dependent upon NOergic signaling in rodents (23-25). Having established the loss of nNOS expression in hypothalamic sites in PAMH mice, we next addressed whether the replenishment of NO levels could rescue normal sexual function in these females. Control and PAMH female mice were injected either with saline or S-nitroso-N-acetylpenicillamine (SNAP; 8 mg/kg), a well-known NO donor to boost circulating NO levels. Saline-injected PAMH mice displayed the previously observed impairment in lordosis behavior in comparison with saline-injected controls (FIG. 3A; P<0.0001). The administration of SNAP prior to testing robustly recovered normal lordosis behavior in PAMH female mice similar to control levels (FIG. 3A; effect of SNAP treatment: P<0.0001, F1, 34=52.29). The recovery of lordosis behavior reached an approximated 7.28-fold increase in PAMH female mice following SNAP treatment (LQPAMH+saline=9.37±2.48 vs. LQPAMH+SNAP=68.17±6.97). Concurrently, SNAP administration significantly reduced both mild (FIG. 3B; P<0.0001) and severe (FIG. 3C; P<0.0001) rejection behaviors in PAMH female mice similar to control levels, while it did not change these parameters in control females (effect of SNAP treatment for MRQ: P<0.0001, F1, 34=27.71; effect of SNAP treatment for SRQ: P<0.0001, F1, 34=40.47).

We aimed to determine how 8 mg/kg SNAP treatment may change the neuronal activation landscape in the female brain in estrus control and PAMH mice following the lordosis test. We chose to evaluate the percentage of cells expressing cFOS, an immediate early gene product and proxy of enhanced neuronal activity, in hypothalamic and limbic areas, which share either direct or indirect connectivity with the VMH (25, 54, 55) and are involved in the control of both appetitive and consummatory aspects of sexual behavior (51-53, 56): RP3V, MPA, MnPO, aBNST, ARN, pdMeA, dorsomedial hypothalamus (DMH), ventrolateral and dorsomedial periaqueductal grey matter (vlPAG and dmPAG, respectively), and ventral tegmental area (VTA) (Data not shown). The percentage of cells expressing cFOS was also examined within the vlVMH and dorsomedial (dVMH) subregions of the VMH (Data not shown). We found that vehicle-treated estrus PAMH female mice had a significantly lower percentage of cFOS expression within the MPA, MnPO, vlVMH, and pdMeA compared with control animals either treated with vehicle or SNAP (P<0.05; Data not shown). Remarkably, SNAP administration increased cFOS expression in all of these four regions in PAMH mice to control levels (Data not shown). In both regions of the PAG, SNAP-treated control mice displayed lower cFOS expression compared with vehicle-treated control mice (P<0.05), whereas SNAP treatment did not alter cFOS expression in these regions in either PAMH mice groups (Data not shown). Evaluation of the proportion of nNOS neurons expressing cFOS in each of these areas revealed that only a small portion of nNOS neurons express cFOS in vehicle-treated PAMH mice in the vlVMH (7.92±4.88%) and pdMeA (3.78±3.40%) following lordosis test. SNAP-treatment of PAMH mice fully restored the proportion of nNOS cells expressing cFOS to control levels (P<0.05; Data not shown). These results indicate that SNAP-mediated amelioration of sexual dysfunction in PAMH female mice may result from the recovery of normal neuronal activation in hypothalamic and limbic structures in the female brain involved in sexual behavior.

NO-mediated physiological responses are mostly dictated by intracellular levels of cyclic guanosine monophosphate (cGMP). Commonly used drugs for male sexual dysfunction, such as sildenafil citrate, commercialized as Viagra®, enhance NOergic signaling by decreasing the degradation of cGMP accumulating intracellular levels of the latest. To determine the severity of NO insufficiency in PAMH mice, we administered sildenafil citrate to both groups and evaluated lordosis behavior. Sildenafil treatment did not rescue normal lordosis behavior in PAMH mice (FIG. 3D; effect of sildenafil treatment: P=0.45, F1, 28=0.57). We also observed that sildenafil treatment was not effective to attenuate either mild (FIG. 3E; P=0.55) or severe (FIG. 3F; P=0.48) rejection behaviors in PAMH mice. Together, our results point to severe NO deficiency in PAMH mice and suggest a novel therapeutic approach to rescue normal sexual function in PCOS based on enhancing NO production rather than controlling the downstream cGMP levels.

Chemogenetic Silencing of nNOS^{VMH} Neurons Recapitulates PCOS-Like Sexual Dysfunction.

Among the three hypothalamic areas investigated in our study, the VMH (nNOS^{VMH}) is considered as the NOergic output responsible to control sexual performance in mammalian females (23), whereas nNOS^{RP3V} and nNOS^{ARN} are likely to contribute to the neuroendocrine control of GnRH/LH secretion (57, 58). PR^{VMH} neurons are also known to participate in female sexual behavior (20), and here, we demonstrated that about 60% of the nNOS^{VMH} neuronal population co-express PR. Hence, we focused our investigations to examine whether acute chemogenetic inhibition of nNOS^{VMH} neurons would promote the impairment of sexual function such as the observed in PAMH female mice. To this end, we selectively targeted the expression of designer receptors exclusively activated by designer drugs (DREADD)-based tool hM4(Gi) into the VMH of Nos1cre/+ (Nos1-ires-cre; B6.129-Nos1tm1(cre)Mgmj/J) female mice (59). Viral transduction was established using a Cre-recombinase-dependent adeno-associated virus serotype 9 in which hM4 (Gi) is fused with mCherry reporter under the control of human synapsin (hSyn) promoter (AAV9-hSyn-DIO-hM4D(Gi)-mCherry) (Data not shown). The control group was composed of wild-type female mice receiving the same stereotaxic surgery. This approach provided an average efficiency of 86.82±2.33% of nNOS^{VMH} neurons targeted with AAV9-hM4 (Gi)-mCherry and with an off-target infection rate of 5.48±2.17% in non-nNOS^{VMH} neurons (N=5; Data not shown).

Following viral transfection, Nos1^{cre/+} female mice were subjected to two lordosis tests and randomly received either vehicle or the DREADD ligand clozapine N-oxide (CNO; 3 mg/kg) one hour before behavioral assessment. We discovered that acute inhibition of nNOS^{VMH} neurons with CNO markedly impaired lordosis behavior (FIG. 3G; LQvehicle=67.39±6.13 vs. LQCNO=4.24±2.26; P<0.01; Wilcoxon signed-rank test) compared with vehicle injection. Over the same assessment frame, CNO-injected mice displayed higher mild (FIG. 3H; P<0.01) and severe (FIG. 3I; P<0.01) rejection behaviors than vehicle-injected mice. The control group did not show any difference between treatments when analyzing either lordosis (Data not shown), mild rejection (Data not shown), or severe rejection behaviors (Data not shown).

Our data indicate that selective inhibition of nNOS^{VMH} neurons recapitulates the observed sexual dysfunction and enhancement of rejection behaviors of our PCOS pre-clinical mouse model. These results also reveal that nNOS^{VMH} neurons are functionally required for the display of female sexual behavior in mice.

Kisspeptin does not Rescue Normal Sexual Behavior Following Acute Inhibition of nNOS^{VMH} Neurons.

Recent reports support the idea that kisspeptin, widely known for its role in the neuroendocrine control of fertility, stimulates female sexual behavior (24, 25). It is suggested that this kisspeptidergic drive might be upstream to nNOS^{VMH} neurons and located in the RP3V region (Kiss1^{RP3V}) (25). However, the RP3RV is also a target from progesterone receptor (PR)-expressing VMH (PR^{VMH}) neurons triggering female sexual behavior in an estrus-dependent manner (15). Here, we aimed to dissect the requirement of the coupling kisspeptin-nNOS^{VMH} neurons for the display of normal sexual behavior and whether disruption of this pathway recapitulates PCOS-like sexual dysfunction.

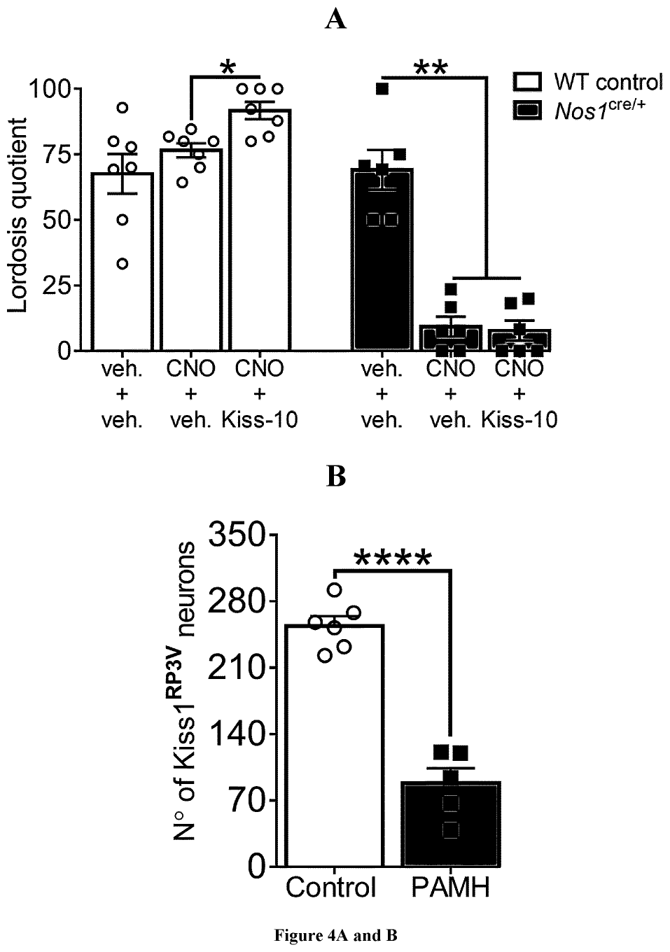

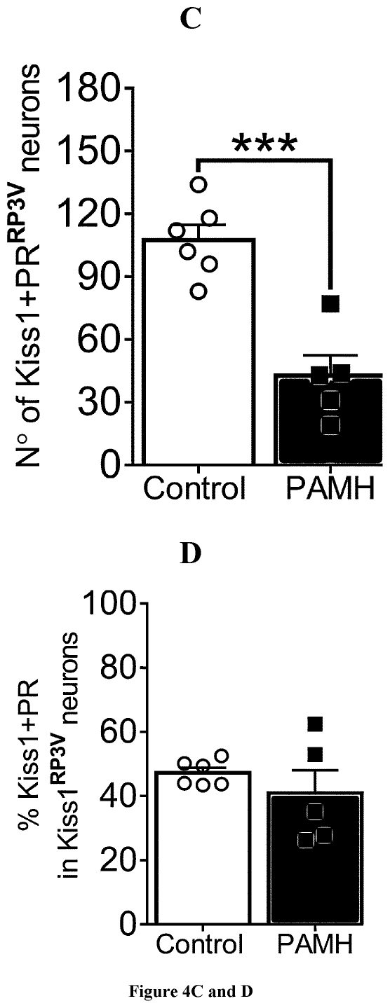

Using a similar approach as described above, we stereotaxically delivered the expression of AAV9-hM4(Gi)-mCherry in nNOS^{VMH} neurons in both Nos1cre/+ and wild-type female mice. Following viral transfection, all females were sexually experienced following three lordosis behavior testing before commencing treatments. Each animal was subjected to different treatments applying a pre-injection (−60 min) with either vehicle or CNO followed by injection (−30 min) with vehicle or kisspeptin (kisspeptin-10; 0.52 μg/kg) before lordosis test (Data not shown). In control female mice, CNO alone did not have any effect on the behavioral output whilst kisspeptin injection significantly enhanced lordosis performance (FIG. 4A; P<0.05; one-way repeated measures ANOVA with Tukey's post hoc test). In Nos1cre/+ female mice, pre-treatment CNO caused significant disruption of sexual behavior and kisspeptin injection was not able to reverse this condition (FIG. 4A; P<0.01; one-way repeated measures ANOVA with Tukey's post hoc test).

Previously, our group showed that a certain degree of masculinization occurs within different hypothalamic nuclei in PAMH female mice (30). Here, we examined whether PAMH treatment affects progesterone-sensitive Kiss1^{RP3V} neurons. Using the reporter knock-in mouse line Kiss1-(IRES)-Cre^{+/-}/ROSA26-CAGS-tauGFP^{+/-} (Kiss1-GFP), in which the expression of Cre recombinase enzyme is dependent upon kisspeptin promoter (60), we generated control and PAMH female mice (Data not shown). This approach allows proper visualization of Kiss1 cell bodies, which is commonly challenging with traditional immunohistochemical approaches. Neuroanatomical analysis revealed that PAMH female mice have a significantly lower number of Kiss1^{RP3V} neurons when compared with control female mice (Data not shown; 65.3% of reduction; P<0.0001). We could also detect a disruption in the number of Kiss1^{RP3V} neurons expressing PR in PAMH female mice in comparison with control females (Data not shown; P<0.001), although both groups presented a similar percentage of PR-expressing Kiss1^{RP3V} neurons (FIG. 4B). Further investigation revealed that kisspeptin and nNOS are mostly expressed in two distinct populations within the RP3V region (Data not shown) whereas only a small portion of neurons express both proteins similarly in control and PAMH female mice (Data not shown; control=0.62% vs. PAMH=0.83%).

These data suggest that kisspeptin signaling is upstream nNOS^{VMH} neuron-mediated lordosis behavior, and that conjoint disrupted expression of both Kiss1^{RP3V} and nNOS^{VMH} might contribute to sexual dysfunction in PCOS.

### DISCUSSION

There is increasing recognition of the detrimental impact of PCOS on health-related quality of life. Among these health burdens, clinical studies identified the presence of impaired sexual function in nearly one-third of young women with PCOS (6, 8). Recent broad meta-analysis investigation evaluating eighteen studies, comprising about 3,900 patients worldwide, shows that women with PCOS have significant sexual dysfunction, estimated as decreased sexual satisfaction, arousal, lubrication, and orgasm (8). This evidence strongly points to central impairments in the brain of women with PCOS.

The employment of pre-clinical models offers the unique opportunity to investigate the etiology of sexual dysfunction in PCOS. PAMH female mice have recently been used to understand neuroendocrine impairments (30, 61), infertility (30), and transgenerational epigenetic inheritance of the disease (31). Here, we confirm that PAMH modeling is a robust translational tool to investigate reproductive dysfunction in PCOS. We report for the first time that androgen excess and high LH secretion, a proxy of high GnRH secretion, are also present over the dark phase of the light-dark cycles, when mice are most receptive for sexual intercourse and when we performed the behavioral experiments. Notably, we detected higher testosterone levels in PAMH females in estrus over the dark phase as compared to our previous reports (30) and others (62), in which diestrous mice were characterized only over the light phase. It remains unknown how precisely testosterone levels fluctuate over the estrous cycle and light-dark cycles in control and PCOS-like mice; however, these observations suggest that high testosterone levels during the estrous phase might disarray progesterone-mediated actions in hypothalamic sex circuits of PCOS-like mice (27, 63, 64).

Our study reveals the presence of severe alterations in the display of mating behavior in PAMH mice such as impaired lordosis and male-directed preference. Notably, impairment in lordosis behavior and enhanced sexual rejection were observed in both intact and in ovariectomized PAMH females supplemented with E2+P4. This suggests that intrinsic defects may arise from developmental disruptions in the PCOS-like condition most likely impinging on central ovarian steroid hormone-sensitive neurons. Consistent with this hypothesis, we documented here that PCOS-like female mice have a robust loss of PR-expressing neurons in the VMH, which may profoundly affect the social interaction between mating partners and sexual performance. It is classically known that progesterone signaling through its classical pathway via PR is required for the full display of lordosis behavior (40, 43). This requirement is placed in the hypothalamic module controlling sexual behavior in mammals, the VMH (15, 19, 20, 22, 44). PRVMH neurons are a robust and required component of the female sexual brain for proper mating behavior (20). PR^{VMH} neurons are primed by estrogen and structural circuit changes strengthening the connection between these neurons and their output within the RP3V region of the hypothalamus when female mice are receptive (15). This circuit plasticity is accompanied by an increase in the activity of PRVMH neurons during both mating chemo-investigation and the display of lordosis (15). This shows that PRVMH neurons are a functionally relevant component of female sex circuits and that, loss of PR expression in the VMH may highly contribute to sexual dysfunction in PCOS. Interestingly, among the three hypothalamic nuclei analyzed, the VMH contained the highest colocalization rate between PR and nNOS, with nearly 90% of PR cells co-expressing nNOS in control and PAMH mice, as opposed to 30-40% in the RP3V and only 5% in the ARN. These data further point to the VMH as a «hot spot» for NO signaling in progesterone-sensitive cells of the hypothalamus.

We provide evidence that there is a great loss of progesterone-sensitive nNOS^{VMH} neurons in PAMH female mice compared to controls. Progesterone actions in sex circuits are enhanced by drugs that increase cGMP levels, being a critical molecular output of NO actions in neuronal circuits (65, 66). The NO-cGMP pathway is well-characterized to mediate lordosis in rodents (23) but, to date, it was unclear the functional relevance of nNOS^{VMH} neurons for this behavior. We showed that treatment with sildenafil citrate, which impedes the degradation of cGMP, is not sufficient to promote the recovery of normal lordosis in PAMH mice demonstrating the severity of NO deficiency in the VMH. A major finding from our study is that nNOS^{VMH} neurons are required to drive normal sexual behavior in female mice and that silencing these neurons using chemogenetics also enhances sexual rejection behaviors. Sexual rejection is profoundly modulated by sensorial inputs from vomeronasal organ (VNO) pathways in females (42, 51). For instance, the pheromone ESP22, which negatively affects lordosis, activates VNO-dependent inhibitory GABAergic input to the vlVMH enhancing female sexual rejection (42). This evidence is in parallel with our findings illustrating the great importance of proper vlVMH neuronal activity in female proceptive behavior. We support the idea that loss of nNOS^{VMH} neurons may impair the integration of sensorial inputs arriving in the VMH leading to prolonged sexual rejection in PCOS-like mice, but whether these VNO pathways directly regulate nNOS^{VMH} neuronal activity remains to be investigated. GABAergic transmission within the VMH has been reported to facilitate lordosis in female rodents (67), and possibly projections from pdMeA GABA neurons to the vlVMH might mediate this facilitatory role (68). However, recent data shows that neuroendocrine dysfunction is highly associated with enhanced GABAergic inputs (30, 64, 69) and signaling (27, 70) in the hypothalamus in PCOS mouse models, and high levels of GABA in the cerebrospinal fluid of PCOS women (71). Although it is tempting to speculate that excessive GABAergic transmission may override the facilitatory role of GABA in the VMH and be detrimental to lordosis behavior, future research should determine a definitive role of GABAergic pathways controlling female sexual behavior in the context of PCOS specifically.

Among its various afferents, Kiss1^{RP3V} neurons have been recently proposed to contact the vlVMH to regulate female sexual behavior in mice (25). However, it is also known that PRVMH neurons project back to the RP3V displaying an estrous cycle-dependent functional connectivity between both nuclei. Here, using a pharmacological mechanistic approach, we showed that kisspeptin-mediated sexual effects are only relevant and dependent upon the proper function of nNOS^{VMH} neurons. This strengthens the idea that Kiss1^{RP3V} neurons are placed upstream of nNOS^{VMH} neurons to drive lordosis behavior. However, we cannot exclude the possibility that nNOS^{VMH} neurons also impinge upon Kiss1^{RP3V} neuronal activity, forming a homeostatic loop between the RP3V and the VMH over the control of female sexual behavior. Further dissection showed that PAMH female mice present a robust reduction of progesterone sensitive-Kiss1^{RP3V} neurons, which most likely contribute to the loss of kisspeptin-nNOS^{VMH} coupling required for proper sexual function in PCOS.

We currently do not know whether this reduction of nNOS-immunoreactivity results from cell-death events occurring during early life, when these hypothalamic circuits are actively shaped, or whether it is the result of epigenetic mechanisms impinging on the expression nNOS in PCOS-like animals. Based on our previous works, we can speculate that exposure to high AMH levels in utero leads to developmental reprogramming of the female fetuses' brain promoting cytoarchitectural remodeling of hypothalamic circuits involved in sexual functions over adult life (30). Our previous study had primarily shown that AMH may act both in the maternal brain circuitry and placental tissue enhancing androgen production through neuroendocrine and local modulation, respectively (30). Recent studies suggest that the PAMH-induced reproductive phenotype is mediated by androgen signaling at the level of kisspeptin cells (61), and functional androgen receptor (AR) in neurons is required for the presentation of PCOS-like features in female mice (72). In addition, previous works have shown that exposure to high androgen levels lowers the hypothalamic expression of nNOS (73) and that AR signaling and its interactions with estrogen receptor alpha (ERα) signaling have profound organizational effects on the number of nNOS neurons in limbic areas in mice (50, 74, 75). Consistently, we found that PAMH females present a loss of nNOS expression in the hypothalamic R3PV, VMH, and ARN. However, we did not detect any significant changes in the number of nNOS-expressing neurons distributed in other hypothalamic and limbic regions of PAMH brains as compared to control animals, suggesting that such loss is region-specific and that the lack of NO in these areas would disrupt normal neurotransmitter function and impact sexual behavior.

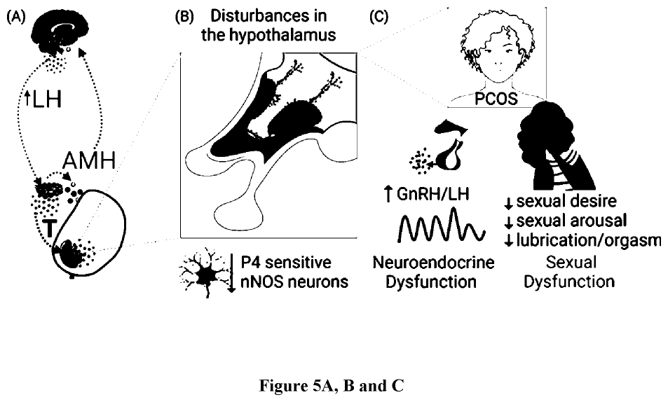

We hypothesize that in PCOS-like animals, perinatal exposure to androgen excess has organizational actions on nNOS-ir cell numbers in hypothalamic areas associated with the control of reproductive functions and sexual behavior, thus triggering developmental brain circuit abnormalities with detrimental outcomes over adult reproductive life and finally affecting proper female sexual performance and neuroendocrine functions (FIG. 5).

We also report, in PAMH female mice, a profound reduction of progesterone-sensitive nNOS neurons within the RP3V and ARN, which are critical sites for the neuroendocrine control of GnRH neuron function and fertility, in PAMH female mice for the first time. Our neuroanatomical data were also associated with the detection of high LH pulses in estrous PAMH mice implying that the loss of homeostatic control of the GnRH/LH secretion in PAMH may be linked to impaired progesterone signaling at the central level as previously documented in PCOS women (36, 76, 77) and other PCOS models (64).

The present investigation also revisited the expression of Kiss1^{RP3V} neurons in PAMH female mice and documented that these mice have a profound loss of progesterone-sensitive Kiss1^{RP3V} neurons. This particular group of Kiss 1RP3T neurons is pivotal to the pre-ovulatory GnRH/LH surge (78, 79), and disruptions in this population are likely to contribute to fertility problems in a PCOS-like condition

The disruption of NO signaling in the RP3V and ARN of PAMH females may be related to the control of GnRH neuron function as previously discovered by our group (62, 63) and others (64, 65). In addition, the present investigations revisited the expression of Kiss1^{RP3V} neurons in PAMH female mice and documented that these mice have a profound loss of progesterone-sensitive Kiss1^{RP3V} neurons. This particular group of Kiss1RP3V neurons is pivotal to the pre-ovulatory GnRH/LH surge (66, 67), and disruptions in this population are likely to contribute to fertility problems in a PCOS-like condition.

The disruption of NO signaling in the RP3V and ARN of PAMH females may be related to the control of GnRH neuron function as previously discovered by our group (80, 81) and others (82, 83). Indeed, NO inhibits the firing behavior of GnRH neurons and it is thought to modulate the bursting firing patterns of these neurons (80). Importantly, PAMH animals present aberrant GnRH neuronal activity together with exacerbated LH pulse secretion (30), and we now provide evidence of a strong reduction in the number of nNOS expressing neurons in these animals, which could account for the accelerated GnRH/LH pulse frequency. Future works are thus required to assess whether NO supplementation may restore the neuroendocrine and reproductive alterations of PCOS-like animals. These investigations are supported by clinical meta-analysis data from 465 PCOS patients showing that this disease is strongly associated with low serum or plasma levels of NO metabolites (45, 46), although it remains unknown whether an attenuated NO pathway is present in PCOS brain circuits. Notably, pharmacological strategies that increase NO levels have been used in the clinics to treat women with PCOS and they have been shown to hold promising therapeutic potential for the reproductive alterations of PCOS as they improved menstrual cyclicity, ovulation (84, 85), and pregnancy rates (85) in those women.

Here, we provide new evidence that the use of NO donors (SNAP) ameliorates sexual dysfunction in a pre-clinical PCOS model. Changes in cFOS expression within the hypothalamic and limbic systems driven by SNAP administration suggest that NO supplementation may recover sexual function by reinstating normal circuitry response to a sexual encounter in our model. It will be interesting in the future to determine whether women with PCOS or women affected by hypoactive sexual desire disorder (HSDD), the most severe form of low sexual drive (86), present altered central levels of NO and/or nNOS circuitry, and whether they could eventually benefit from such therapeutic options. To date, current treatments for HSDD are limited to two US Food and Drug Administration-approved medications for premenopausal women. Flibanserin, a serotonin mixed agonist and antagonist (87), and bremelanotide, a melanocortin 4 receptor (MC4R) agonist (88). However, the efficacy of these treatments, their tolerability, and the primary beneficial outcomes of the clinical trials are controversial (89-92). Therefore, the discovery of novel therapeutic strategies for female sexual dysfunction remains an urgent need in clinics.

In conclusion, our results have broad repercussions for our understanding of developmental reprogramming in the female brain leading to disturbances in hypothalamic circuitry driving sexual dysfunction in PCOS. These findings provide grounds for prospective venues toward the discovery of new brain pathways involved in the control of female sexual behavior and the implications of aberrations in these circuits for women's health.

TABLE 1
Number of nNOS neurons in hypothalamic and limbic regions involved
in the control of sexual behaviors in control and PAMH female mice.
No of nNOS neurons

| Nucleus/area | Control | PAMH | P |
|---|---|---|---|
| RP3V | 289.3 ± 8.52 | 129.0 ± 15.23 | <0.0001 |
| vIVMH | 266.4 ± 23.38 | 147.9 ± 7.52 | 0.0005 |
| ARN | 240.2 ± 7.44 | 153.8 ± 10.66 | 0.0002 |
| MPA | 155.6 ± 18.15 | 178.8 ± 38.27 | 0.599 |
| MnPO | 219.2 ± 66.11 | 266.4 ± 92.89 | 0.689 |
| aBNST | 426.0 ± 76.03 | 309.6 ± 68.57 | 0.288 |
| pdMeA | 208.4 ± 64.25 | 173.6 ± 35.23 | 0.647 |

The table shows that estrus control and PAMH female mice show similar number of nNOS neurons in the rostral periventricular area of the third ventricle (RP3V), ventrolateral aspect of the ventromedial hypothalamus (vlVMH), arcuate nucleus (ARN), medial preoptic area (MPA), median preoptic nucleus (MnPO), anterior division of the bed nucleus of the stria terminalis (aBNST), and posterodorsal medial amygdala nucleus (pdMeA). P value indicated in the table was obtained following statistical analysis using two-tailed unpaired Student's t test. Values are shown as mean±SEM.

### REFERENCES

Throughout this application, various references describe the state of the art to which this invention pertains. The disclosures of these references are hereby incorporated by reference into the present disclosure.
1. D. Lizneva, et al., Criteria, prevalence, and phenotypes of polycystic ovary syndrome. Fertil. Steril. 106, 6-15 (2016).
2. R. Azziz, et al., Polycystic ovary syndrome. Nat. Rev. Dis. Prim. 2, 16057 (2016).
3. Rotterdam ESHRE/ASRM-Sponsored PCOS Consensus Workshop Group, Revised 2003 consensus on diagnostic criteria and long-term health risks related to polycystic ovary syndrome. Fertil. Steril. 81, 19-25 (2004).
4. D. A. Dumesic, et al., Scientific statement on the diagnostic criteria, epidemiology, pathophysiology, and molecular genetics of polycystic ovary syndrome. Endocr. Rev. 36, 487-525 (2015).
5. A. H. Rellini, et al., Differences in sexual desire between women with clinical versus biochemical signs of hyperandrogenism in polycystic ovarian syndrome. Horm. Behav. 63, 65-71 (2013).
6. M. Steinberg Weiss, et al., Lifestyle modifications alone or combined with hormonal contraceptives improve sexual dysfunction in women with polycystic ovary syndrome. Fertil. Steril. 115, 474-482 (2021).
7. D. W. Stovall, J. L. Scriver, A. H. Clayton, C. D. Williams, L. M. Pastore, Sexual function in women with polycystic ovary syndrome. J. Sex. Med. 9, 224-230 (2012).
8. H. Pastoor, et al., Sexual function in women with polycystic ovary syndrome: a systematic review and meta-analysis (2018) https:/doi.org/10.1016/j.rbmo.2018.09.010 (May 8, 2020).
9. X. Tian, et al., Sexual Function in Chinese Women with Polycystic Ovary Syndrome and Correlation with Clinical and Biochemical Characteristics. Reprod. Sci. (2021) https:/doi.org/10.1007/s43032-021-00612-4 (Jul. 1, 2021).
10. J. G. Pfaus, S. L. Jones, L. M. Flanagan-Cato, J. D. Blaustein, Female Sexual Behavior (2015).
11. K. M. Lenz, M. M. McCarthy, Organized for sex-steroid hormones and the developing hypothalamus. Eur. J. Neurosci. 32, 2096-2104 (2010).
12. R. B. Simerly, Wired for reproduction: Organization and development of sexually dimorphic circuits in the mammalian forebrain. Annu. Rev. Neurosci. 25, 507-536 (2002).
13. C. A. Cornil, G. F. Ball, J. Balthazart, T. D. Charlier, Organizing effects of sex steroids on brain aromatase activity in quail. PLOS One 6 (2011).
14. K. Chachlaki, J. Garthwaite, V. Prevot, The gentle art of saying NO: How nitric oxide gets things done in the hypothalamus. Nat. Rev. Endocrinol. 13, 521-535 (2017).
15. S. Inoue, et al., Periodic Remodeling in a Neural Circuit Governs Timing of Female Sexual Behavior. Cell 179, 1393-1408.e16 (2019).
16. B. Zempo, T. Karigo, S. Kanda, Y. Akazome, Y. Oka, Morphological analysis of the axonal projections of EGFP-labeled ESR1-expressing neurons in transgenic female medaka. Endocrinology 159, 1228-1241 (2018).
17. K. Sinchak, et al., Modulation of the arcuate nucleus-medial preoptic nucleus lordosis regulating circuit: a role for GABAB receptors. Horm. Behav. 64, 136-43 (2013).
18. D. Daniels, L. M. Flanagan-Cato, Functionally-defined compartments of the lordosis neural circuit in the ventromedial hypothalamus in female rats. J. Neurobiol. 45, 1-13 (2000).
19. K. Nomoto, S. Q. Lima, Enhanced male-evoked responses in the ventromedial hypothalamus of sexually receptive female mice. Curr. Biol. 25, 589-594 (2015).
20. C. F. Yang, et al., Sexually dimorphic neurons in the ventromedial hypothalamus govern mating in both sexes and aggression in males. Cell 153, 896-909 (2013).
21. D. W. Kim, et al., Multimodal Analysis of Cell Types in a Hypothalamic Node Controlling Social Behavior. Cell 179, 713-728.e17 (2019).
22. D. W. Pfaff, Y. Sakuma, Facilitation of the lordosis reflex of female rats from the ventromedial nucleus of the hypothalamus. J. Physiol. 288, 189-202 (1979).
23. S. K. Mani, et al., Nitric oxide mediates sexual behavior in female rats. Proc. Natl. Acad. Sci. U.S.A 91, 6468-6472 (1994).
24. Y. Bentefour, J. Bakker, Kisspeptin signaling and nNOS neurons in the VMHvl modulate lordosis behavior but not mate preference in female mice. Neuropharmacology 198, 108762 (2021).
25. V. Hellier, et al., Female sexual behavior in mice is controlled by kisspeptin neurons. Nat. Commun. 9, 400 (2018).
26. O. González-Flores, A. M. Etgen, The nitric oxide pathway participates in estrous behavior induced by progesterone and some of its ring A-reduced metabolites. Horm. Behav. 45, 50-57 (2004).
27. S. D. Sullivan, S. M. Moenter, Prenatal androgens alter GABAergic drive to gonadotropin-releasing hormone neurons: implications for a common fertility disorder. Proc Natl Acad Sci USA 101, 7129-7134 (2004).
28. H. Hedström, et al., Women with polycystic ovary syndrome have elevated serum concentrations of and altered GABAA receptor sensitivity to allopregnanolone. Clin. Endocrinol. (Oxf). 83, 643-650 (2015).
29. M. S. B. Silva, M. Prescott, R. E. Campbell, Ontogeny and reversal of brain circuit abnormalities in a preclinical model of PCOS. JCI insight 3, pii: 99405 (2018).
30. B. Tata, et al., Elevated prenatal anti-Müllerian hormone reprograms the fetus and induces polycystic ovary syndrome in adulthood. Nat. Med. 24, 834-846 (2018).
31. N. E. H. Mimouni, et al., Polycystic ovary syndrome is transmitted via a transgenerational epigenetic process. Cell Metab. 33, 513-530.e8 (2021).
32. T. T. Piltonen, et al., Circulating antimüllerian hormone and steroid hormone levels remain high in pregnant women with polycystic ovary syndrome at term. Fertil. Steril. 111, 588-596.el (2019).
33. L. Detti, et al., Serum Anti-Müllerian hormone (AMH) in mothers with polycystic ovary syndrome (PCOS) and their term fetuses. Syst. Biol. Reprod. Med. 65, 147-154 (2019).
34. E. Stener-Victorin, et al., Animal Models to Understand the Etiology and Pathophysiology of Polycystic Ovary Syndrome. Endocr. Rev. 41 (2020).
35. T. L. Daniels, S. L. Berga, Resistance of gonadotropin releasing hormone drive to sex steroid-induced suppression in hyperandrogenic anovulation. J. Clin. Endocrinol. Metab. 82, 4179-4183 (1997).
36. A. E. Taylor, et al., Determinants of abnormal gonadotropin secretion in clinically defined women with polycystic ovary syndrome. J. Clin. Endocrinol. Metab. 82, 2248-2256 (1997).
37. R. Azziz, et al., The Androgen Excess and PCOS Society criteria for the polycystic ovary syndrome: the complete task force report. Fertil. Steril. 91, 456-88 (2009).
38. L. G. Cooney, I. Lee, M. D. Sammel, A. Dokras, High prevalence of moderate and severe depressive and anxiety symptoms in polycystic ovary syndrome: A systematic review and meta-analysis. Hum. Reprod. 32, 1075-1091 (2017).
39. A. A. Walf, C. A. Frye, The use of the elevated plus maze as an assay of anxiety-related behavior in rodents. Nat. Protoc. 2, 322-328 (2007).
40. S. K. Mani, et al., Dopamine requires the unoccupied progesterone receptor to induce sexual behavior in mice. Mol. Endocrinol. 10, 1728-1737 (1996).
41. C. Lenschow, S. Q. Lima, In the mood for sex: neural circuits for reproduction. Curr. Opin. Neurobiol. 60, 155-168 (2020).
42. T. Osakada, et al., Sexual rejection via a vomeronasal receptor-triggered limbic circuit. Nat. Commun. 9, 4463 (2018).
43. J. P. Lydon, et al., Mice lacking progesterone receptor exhibit pleiotropic reproductive abnormalities. Genes Dev. 9, 2266-2278 (1995).
44. G. J. Romano, A. Krust, D. W. Pfaff, Expression and estrogen regulation of progesterone receptor mRNA in neurons of the mediobasal hypothalamus: an in situ hybridization study. Mol. Endocrinol. 3, 1295-1300 (1989).
45. C. Meng, Nitric oxide (NO) levels in patients with polycystic ovary syndrome (PCOS): a meta-analysis. J. Int. Med. Res. 47, 4083-4094 (2019).
46. M. B. Krishna, et al., Impaired Arginine Metabolism Coupled to a Defective Redox Conduit Contributes to Low Plasma Nitric Oxide in Polycystic Ovary Syndrome. Cell. Physiol. Biochem. 43, 1880-1892 (2018).
47. C. L. Pastor, M. L. Griffin-Korf, J. A. Aloi, W. S. Evans, J. C. Marshall, Polycystic ovary syndrome: evidence for reduced sensitivity of the gonadotropin-releasing hormone pulse generator to inhibition by estradiol and progesterone. J. Clin. Endocrinol. Metab. 83, 582-90 (1998).
48. P. B. Chen, et al., Sexually Dimorphic Control of Parenting Behavior by the Medial Amygdala. Cell 176, 1206-1221.e18 (2019).
49. G. C. Panzica, et al., Effects of gonadal hormones on central nitric oxide producing systems. Neuroscience 138, 987-995 (2006).
50. E. M. Scordalakes, S. J. Shetty, E. F. Rissman, Roles of estrogen receptor a and androgen receptor in the regulation of neuronal nitric oxide synthase. J. Comp. Neurol. 453, 336-344 (2002).
51. K. K. Ishii, et al., A Labeled-Line Neural Circuit for Pheromone-Mediated Sexual Behaviors in Mice. Neuron 95, 123-137.e8 (2017).
52. S. da Silva Pacheco, T. Araujo Rondini, J. Cioni Bittencourt, C. Fuzeti Elias, Neurons expressing estrogen receptor a differentially innervate the periaqueductal gray matter of female rats. J. Chem. Neuroanat. 97, 33-42 (2019).
53. L. H. Calizo, L. M. Flanagan-Cato, Hormonal-Neural Integration in the Female Rat Ventromedial Hypothalamus: Triple Labeling for Estrogen Receptor-a, Retrograde Tract Tracing from the Periaqueductal Gray, and Mating-Induced Fos Expression. Endocrinology 144, 5430-5440 (2003).
54. N. S. Canteras, R. B. Simerly, L. W. Swanson, Organization of projections from the ventromedial nucleus of the hypothalamus: A Phaseolus vulgaris-Leucoagglutinin study in the rat. J. Comp. Neurol. 348, 41-79 (1994).
55. L. Lo, et al., Connectional architecture of a mouse hypothalamic circuit node controlling social behavior. Proc. Natl. Acad. Sci. U.S.A 116, 7503-7512 (2019).
56. O. Iyilikci, J. Balthazart, G. F. Ball, Medial preoptic regulation of the ventral tegmental area related to the control of sociosexual behaviors. eNeuro 3 (2016).
57. N. Bellefontaine, et al., Leptin-dependent neuronal NO signaling in the preoptic hypothalamus facilitates reproduction. J. Clin. Invest. 124, 2550-2559 (2014).
58. V. Delli, M. S. B. Silva, V. Prévot, K. Chachlaki, The KING of reproduction: Kisspeptin/nNOS interactions shaping hypothalamic GnRH release. Mol. Cell. Endocrinol. 532, 111302 (2021).
59. R. L. Leshan, M. Greenwald-Yarnell, C. M. Patterson, I. E. Gonzalez, M. G. Myers, Leptin action through hypothalamic nitric oxide synthase-1-expressing neurons controls energy balance. Nat. Med. 18, 820-823 (2012).
60. C. Mayer, et al., Timing and completion of puberty in female mice depend on estrogen receptor a-signaling in kisspeptin neurons. Proc. Natl. Acad. Sci. U.S.A 107, 22693-22698 (2010).
61. E. V. Ho, et al., Reproductive Deficits Induced by Prenatal Antimüllerian Hormone Exposure Require Androgen Receptor in Kisspeptin Cells. Endocrinol. (United States) 162 (2021).
62. A. M. Moore, M. Prescott, R. E. Campbell, Estradiol negative and positive feedback in a prenatal androgen-induced mouse model of polycystic ovarian syndrome. Endocrinology 154, 796-806 (2013).
63. J. Sun, S. M. Moenter, Progesterone treatment inhibits and dihydrotestosterone (DHT) treatment potentiates voltage-gated calcium currents in gonadotropin-releasing hormone (GnRH) neurons. Endocrinology 151, 5349-58 (2010).
64. A. M. Moore, M. Prescott, C. J. Marshall, S. H. Yip, R. E. Campbell, Enhancement of a robust arcuate GABAergic input to gonadotropin-releasing hormone neurons in a model of polycystic ovarian syndrome. Proc. Natl. Acad. Sci. U.S.A 112, 596-601 (2015).
65. R. E. Whalen, A. H. Lauber, Progesterone substitutes: cGMP mediation. Neurosci. Biobehav. Rev. 10, 47-53 (1986).
66. H. P. Chu, J. C. Morales, A. M. Etgen, Cyclic GMP may potentiate lordosis behaviour by progesterone receptor activation. J. Neuroendocrinol. 11, 107-113 (1999).
67. M. M. McCarthy, K. F. Malik, H. H. Feder, Increased GABAergic transmission in medial hypothalamus facilitates lordosis but has the opposite effect in preoptic area. Brain Res. 507, 40-44 (1990).
68. C. S. Johnson, W. Hong, P. E. Micevych, Posterodorsal Medial Amygdala Regulation of Female Social Behavior: GABA versus Glutamate Projections. J. Neurosci. 41, 8790-8800 (2021).
69. M. S. B. Silva, et al., Activation of arcuate nucleus GABA neurons promotes luteinizing hormone secretion and reproductive dysfunction: Implications for polycystic ovary syndrome. EBioMedicine 44, 582-596 (2019).
70. T. Berg, M. A. Silveira, S. M. Moenter, Prepubertal development of GABAergic transmission to gonadotropin-releasing hormone (GnRH) neurons and postsynaptic response are altered by prenatal androgenization. J. Neurosci. 38, 2283-2293 (2018).
71. J. F. Kawwass, K. M. Sanders, T. L. Loucks, L. C. Rohan, S. L. Berga, Increased cerebrospinal fluid levels of GABA, testosterone and estradiol in women with polycystic ovary syndrome. Hum. Reprod. 32, 1450-1456 (2017).
72. A. S. L. Caldwell, et al., Neuroendocrine androgen action is a key extraovarian mediator in the development of polycystic ovary syndrome. Proc. Natl. Acad. Sci. U.S.A 114, E3334-E3343 (2017).
73. R. Singh, S. Pervin, J. Shryne, R. Gorski, G. Chaudhuri, Castration increases and androgens decrease nitric oxide synthase activity in the brain: Physiologic implications. Proc. Natl. Acad. Sci. U.S.A 97, 3672-3677 (2000).
74. M. Edelmann, C. Wolfe, E. M. Scordalakes, E. F. Rissman, S. Tobet, Neuronal nitric oxide synthase and calbindin delineate sex differences in the developing hypothalamus and preoptic area. Dev. Neurobiol. 67, 1371-1381 (2007).
75. M. Martini, et al., Androgen receptors are required for full masculinization of nitric oxide synthase system in rat limbic-hypothalamic region. Horm. Behav. 54, 557-564 (2008).
76. S. Chhabra, et al., Progesterone inhibition of the hypothalamic gonadotropin-releasing hormone pulse generator: evidence for varied effects in hyperandrogenemic adolescent girls. J. Clin. Endocrinol. Metab. 90, 2810-5 (2005).
77. C. A. Eagleson, et al., Polycystic ovarian syndrome: evidence that flutamide restores sensitivity of the gonadotropin-releasing hormone pulse generator to inhibition by estradiol and progesterone. J. Clin. Endocrinol. Metab. 85, 4047-52 (2000).
78. S. B. Z. Stephens, et al., Absent Progesterone Signaling in Kisspeptin Neurons Disrupts the LH Surge and Impairs Fertility in Female Mice. Endocrinology 156, 3091-3097 (2015).
79. M. A. Mohr, L. A. Esparza, P. Steffen, P. E. Micevych, A. S. Kauffman, Progesterone Receptors in AVPV Kisspeptin Neurons Are Sufficient for Positive Feedback Induction of the LH Surge. Endocrinol. (United States) 162 (2021).
80. J. Clasadonte, P. Poulain, J.-C. Beauvillain, V. Prevot, Activation of Neuronal Nitric Oxide Release Inhibits Spontaneous Firing in Adult Gonadotropin-Releasing Hormone Neurons: A Possible Local Synchronizing Signal. Endocrinology 149, 587-596 (2008).
81. N. K. Hanchate, et al., Kisspeptin-GPR54 signaling in mouse No-Synthesizing neurons participates in the hypothalamic control of ovulation. J. Neurosci. 32, 932-945 (2012).
82. R. B. McCosh, et al., Evidence that nitric oxide is critical for LH surge generation in female sheep. Endocrinol. (United States) 161 (2020).
83. S. Constantin, D. Reynolds, A. Oh, K. Pizano, S. Wray, Nitric oxide resets kisspeptin-excited GnRH neurons via PIP2 replenishment. Proc. Natl. Acad. Sci. U.S.A 118 (2021).
84. A. Masha, et al., Prolonged treatment with N-acetylcysteine and L-arginine restores gonadal function in patients with polycistic ovary syndrome. J. Endocrinol. Invest. 32, 870-872 (2009).
85. A. Mahran, A. Abdelmeged, H. Shawki, A. Moheyelden, A. M. Ahmed, Nitric oxide donors improve the ovulation and pregnancy rates in anovulatory women with polycystic ovary syndrome treated with clomiphene citrate: A RCT. Int. J. Reprod. Biomed. 14, 9-14 (2016).
86. R. E. Nappi, et al., Management of hypoactive sexual desire disorder in women: Current and emerging therapies. Int. J. Womens. Health 2, 167-175 (2010).
87. S. M. Stahl, Mechanism of action of flibanserin, a multifunctional serotonin agonist and antagonist (MSAA), in hypoactive sexual desire disorder. CNS Spectr. 20, 1-6 (2015).
88. J. G. Pfaus, A. Sadiq, C. Spana, A. H. Clayton, The Neurobiology of Bremelanotide for the Treatment of Hypoactive Sexual Desire Disorder in Premenopausal Women. CNS Spectr., 1-9 (2021).
89. B. Mintzes, L. Tiefer, L. Cosgrove, Bremelanotide and flibanserin for low sexual desire in women: the fallacy of regulatory precedent. Drug Ther. Bull. 59, 185-188 (2021).
90. J. A. Simon, et al., Long-Term Safety and Efficacy of Bremelanotide for Hypoactive Sexual Desire Disorder. Obstet. Gynecol. 134, 909-917 (2019).
91. G. I. Spielmans, Re-Analyzing Phase III Bremelanotide Trials for “Hypoactive Sexual Desire Disorder” in Women. J. Sex Res. 58, 1085-1105 (2021).
92. L. Jaspers, et al., Efficacy and safety of flibanserin for the treatment of hypoactive sexual desire disorder in women: A systematic review and meta-analysis. JAMA Intern. Med. 176, 453-462 (2016).
93. C. S. Caligioni, Assessing reproductive status/stages in mice. Curr. Protoc. Neurosci., 1-8 (2009).
94. F. J. Steyn, et al., Development of a methodology for and assessment of pulsatile luteinizing hormone secretion in juvenile and adult male mice. Endocrinology 154, 4939-4945 (2013).
95. K. Chachlaki, et al., Phenotyping of nNOS neurons in the postnatal and adult female mouse hypothalamus. J. Comp. Neurol. 525, 3177-3189 (2017).
96. A. E. Herbison, S. X. Simonian, P. J. Norris, P. C. Emson, Relationship of neuronal nitric oxide synthase immunoreactivity to GnRH neurons in the ovariectomized and intact female rat. J. Neuroendocrinol. 8, 73-82 (1996).
97. J. A. Johansen, L. G. Clemens, A. A. Nunez, Characterization of copulatory behavior in female mice: Evidence for paced mating. Physiol. Behav. 95, 425-429 (2008).
98. O. Friard, M. Gamba, BORIS: a free, versatile open-source event-logging software for video/audio coding and live observations. Methods Ecol. Evol. 7, 1325-1330 (2016).
99. T. Sir-Petermann, et al., Increased anti-müllerian hormone serum concentrations in prepubertal daughters of women with polycystic ovary syndrome. J. Clin. Endocrinol. Metab. 91, 3105-3109 (2006).

1. (canceled)

2. (canceled)

3. (canceled)

4. (canceled)

5. A method of preventing or treating a sexual dysfunction associated with hypoactive sexual desire disorder (HSDD) in a subject in need thereof, comprising administering to the subject a therapeutically effective amount of a nitric oxide (NO) agent.

6. The method according to claim 5, wherein the Nitric Oxyde (NO) agent is
inhaled nitric oxide (iNO); or
a nitric oxide donor (NO donor).

7. A pharmaceutical composition comprising a nitric oxide (NO) agent and a pharmaceutically acceptable carrier compounded for use in the treatment or prevention of a sexual dysfunction associated with a Polycystic Ovary Syndrome (PCOS) in a subject of need thereof.

8. The pharmaceutical composition according to claim 7, wherein the NO agent is
inhaled nitric oxide (iNO); or
a nitric oxide donor (NO donor).

9. The pharmaceutical composition according to claim 8, wherein the NO donor is selected from the group consisting of isosorbide dinitrate, L-arginine, linsidomine, minoxidil, nicorandil, nitroglycerin, nitroprusside, nitrosoglutathione, and S-nitroso-N-acetyl-penicillamine (SNAP), sodium nitroprusside, glyceryl trinitrate (GTN), isosorbide mononitrate (ISMN), pentaerythrityl tetranitratethe (PETN), a NONOate and an S-nitrosothiol.

10. A method of preventing or treating a sexual dysfunction associated with Polycystic Ovary Syndrome (PCOS) in a patient in need thereof comprising administering to the patient a therapeutically effective amount of a nitric oxide (NO) agent.

11. The method according to claim 10, wherein the NO agent is
inhaled nitric oxide (iNO); or
a nitric oxide donor (NO donor).

12. The method according to claim 11, wherein the NO donor is selected from the group consisting of isosorbide dinitrate, L-arginine, linsidomine, minoxidil, nicorandil, nitroglycerin, nitroprusside, nitrosoglutathione, and S-nitroso-N-acetyl-penicillamine (SNAP), sodium nitroprusside, glyceryl trinitrate (GTN), isosorbide mononitrate (ISMN), pentaerythrityl tetranitratethe (PETN), a NONOate and an S-nitrosothiol.

13. The method according to claim 12, wherein the NONOate is a diazeniumdiolate.

14. The method according to claim 12, wherein the S-nitrosothiol is S-nitrosoglutathione (GSNO).
